(19) United States
(12) Patent Application Publication
(10) Pub. No.: US 2018/0021166 A1
(43) Pub. Date: Jan. 25, 2018

(54) THERMOREGULATION SYSTEM AND METHOD

(71) Applicant: Physiolab Technologies Limited, Colchester Essex (GB)

(72) Inventor: Nicholas Rose, Colchester Essex (GB)

(21) Appl. No.: 15/516,826

(22) PCT Filed: Jul. 21, 2015
(86) PCT No.: PCT/GB2015/052102

§ 371 Date: Aug. 18, 2017

(30) Foreign Application Priority Data
Jul. 31, 2014 (GB) 1413601.4
Mar. 26, 2015 (GB) 1505218.6

(51) Int. Cl. A61F 7/00; A61H 33/00

(52) U.S. Cl. CPC A61F 7/0053; A61F 7/0085; A61H 33/0095; A61F 2007/0054; A61F 2007/0069; A61F 2007/0091

(57) ABSTRACT

Disclosed is a thermoregulation system, including an output port (54), an input port (62), a thermoregulation fluid supply system, an output pump (52) for pumping thermoregulation fluid out through the output port, an input pump (64) for pumping thermoregulation fluid in through the input port, and a pressure control system. The pressure control system is for selectively operating a pressurising element of a personal thermoregulation pack to apply a pressure independently of operation of the input (64) and output (52) pumps.

### TECHNICAL FIELD

The present invention relates to thermoregulation systems for personal use, for example for applying a thermal treatment to a patient in order to alleviate or cure physical injuries and disorders, and methods therefor.

### BACKGROUND

There are numerous instances where it is desired to effect a thermal treatment on a patient. For example, this may be to treat a physical injury, such as of the muscles, ligaments, tendons and the like. It may also be useful in treating skin injuries, as well as illnesses such as infections and so on as well as to accelerate natural recovery.

Thermal treatments of this type have been known for many years, in their simplest form being ice packs.

More recently, thermoregulation devices have been developed which provide built-in control, for example in which fluid is provided to a cuff or pad with particular feed parameters in order to provide a desired energy transfer to a patient. Examples are disclosed in WO 2013/190337 and WO 2013/190336.

WO2004/006814 discloses apparatus for altering the body temperature of a patient. US2005/0065581 discloses a flexible heat exchanger for medical coding and warming applications.

### SUMMARY OF THE PRESENT INVENTION

The present invention seeks to provide an improved thermoregulation system, assembly, and method.

According to as aspect of the invention, there is provided a thermoregulation system, including an output port for coupling to an input of a personal thermoregulation pack, an input port for coupling to an output of a personal thermoregulation pack, a thermoregulation fluid supply system for supplying thermoregulation fluid to the output port, an output pump for pumping thermoregulation fluid out through the output port, an input pump for pumping thermoregulation fluid in through the input port; and a pressure control system for selectively operating a pressurising element of a personal thermoregulation pack to apply a pressure independently of operation of the input and output pumps.

In embodiments, the thermoregulation fluid supply system includes a thermoregulation supply.

According to an aspect of the invention, there is provided a thermoregulation system, including:
an output port for coupling to an input of a personal thermoregulation pack;
an input port for coupling to an output of a personal thermoregulation pack;
a thermoregulation supply for supplying thermoregulation fluid to the output port;
an output pump for pumping thermoregulation fluid out through the output port;
an input pump for pumping thermoregulation fluid in through the input port; and
a pressure control system for selectively operating a pressurising element of a personal thermoregulation pack to apply a pressure independently of operation of the input and output pumps.

One of the feed parameters that the systems disclosed in WO2013/190337 vary is the volume of thermoregulation fluid in the pack. However, it has been found that, in certain positions or orientations, the fluid in some packs can become trapped and can balloon in the pack. This can mean that the volume of fluid in the pack does not precisely match the desired volume, potentially leading to discrepancies in the amount of energy transfer to or from a patient.

Furthermore, in some known systems, the pressure of a pack onto a patient is controlled by the flow of thermoregulation fluid through the pack. However, controlling the flow of thermoregulation fluid affects the rate of fluid flow past the patient which can have an effect on the amount of energy being delivered to the patient as well as the pressure applied to the patient.

Preferred embodiments of the present invention provide both an output pump for pumping thermoregulation fluid out of the fluid supply system and into the pack, and an input pump for pumping thermoregulation fluid out of the pack and back into the supply system. Preferred embodiments also provide a separate system for selectively applying and varying pressure to a patient. Preferred embodiments of the system can therefore be operated to precisely control the rate of thermoregulation fluid passing the patient independently of the pressure with which the pack is pressed against the patient, thereby enabling the pressure to be controlled independently of the rate of energy being passed through the pack.

References herein to the pressure with which a pack is pressed against a user or a patient can refer to either the total pressure with which the pack is pressed against the user or patient or to the pressure with which the pack is pressed against the user or patient by the pressurising element of the pack.

Preferably, pumping rates of the output and input pumps, and therefore the rate of thermoregulation fluid entering and leaving a pack coupled to the output and input ports, can be controlled independently of each other, meaning that in preferred embodiments the volume of fluid in a pack can be controlled precisely. Typically, the volume of thermoregulation fluid in a pack is the integral of the rate of thermoregulation fluid flow into the pack minus the integral of the rate of thermoregulation fluid flow out of the pack, meaning the volume in the pack can be reduced by pumping out at a greater rate than pumping in and the volume can be increased by pumping in at a greater rate than pumping out.

Additionally, in preferred embodiments, the volume of thermoregulation fluid in a pack can be controlled independently of the rate of thermoregulation fluid flowing into the pack. This is because the change in volume of the pack is dependent on the difference between the rates flowing in and out, and not on the absolute rates of flow.

Too much thermoregulation fluid can burn a patient, while too little fluid can cause a pack to be ineffective. Accurate control and adjustment of the volume independently of the pressure with which the pack is pressed against a patient, the rate of thermoregulation fluid flow into the pack, and the temperature of the thermoregulation fluid entering the pack, means that preferred embodiments of the system can respond to correct an undesired pack volume without needing to affect other feed or operation parameters.

Furthermore, during treatment, there will be a variability to a patient's resistance to thermal transfer. This means that in order to maintain a constant thermal exchange between the pack and the patient, it is often necessary to vary one or more operation or feed parameters. The independence of the variability of the operation or feed parameters in embodiments of the invention, means that an appropriate change can be made to the relevant operation or feed parameters while holding the others constant. For example, a volume of fluid in the pack can be varied while keeping the pressure with which the pack is pressed against a patient constant.

Furthermore, embodiments of the invention can compensate for a changing human load and deliver constant pressure while maintaining a constant average temperature of a coupled pack and/or while maintaining a constant volume of thermoregulation fluid in a coupled pack.

In preferred embodiments, the pressure with which the pack is pressed against a patient, the rate of flow of thermoregulation fluid into the pack, the volume of thermoregulation fluid in the pack, and the temperature of thermoregulation fluid entering the pack can each be independently controlled, allowing the system to operate precisely to desired operation or feed parameters.

The rate of flow, the temperature, and other feed parameters of the thermoregulation fluid entering the pack are also preferably the rate of flow, the temperature, and other feed parameters, of thermoregulation fluid out of the output port.

Having an input pump which actively pumps fluid out of the pack means that unwanted air can be pumped out of the pack to make way for thermoregulation fluid. Air is an insulator, meaning that it can interfere with energy transfer to and from the thermoregulation pack. Furthermore, air in the thermal chamber of the pack takes up space that could be occupied by thermoregulation fluid, thereby affecting the performance of the pack. In prior art packs, it was often necessary to have a restricted liquid return to function as an exhaust to allow air to escape from the pack. While a restricted liquid return can be used in embodiments of the invention, it is not necessary in all embodiments since the input pump can forcibly draw air out of the pack.

Preferably, the pressure control system is for selectively varying a pressure applied by a pressurising element of a personal thermoregulation pack independently of operation of the input and output pumps.

Preferably, the pressure control system includes a pressure fluid outlet port for coupling to a pressurising element, preferably a pressure chamber, of a personal interface pack and a pressure fluid pump for pumping pressure fluid into or out of the pressure fluid outlet port.

Preferably, during thermal treatment, the pressure control system is configured to operate a pressurising element of a coupled pack at a lower pressure than thermoregulation fluid in a thermoregulation zone of a coupled pack.

Preferably, the pressure fluid is air.

In preferred embodiments, the thermoregulation fluid is incompressible, preferably a liquid, preferably water.

Having a separate pressure control system means that in preferred embodiments the system can cause a pack to apply even treatment to an area of a patient while adjusting the energy provided to that area independently. It can also mean that only a thin layer of thermoregulation fluid needs to be provided adjacent to the patient by the pack as the pressure is provided by a separate element.

In some embodiments, the input pump is for pumping thermoregulation fluid in through the input port to the thermoregulation supply or supply system.

Preferably, the thermoregulation system includes a control unit, which could be referred to as an operation control unit, operable to control the input and output pumps and the pressure control system. Preferably, the control unit is operable to control the pressure fluid pump and any other controllable components.

The control unit can be operable to control the pumping rates of the input and output pumps independently of each other.

Preferably, the control unit is operable to control, independently of each other, a pressure with which a coupled pack is pressed against a user, a rate of flow of thermoregulation fluid out of the output port, a rate of flow of thermoregulation fluid in through the input port, and a temperature of thermoregulation fluid supplied through the output port.

Preferably, the thermoregulation system or thermoregulation supply or supply system includes a heat control unit for providing thermoregulation fluid at a predetermined thermoregulation temperature.

In some embodiments, the heat control unit is operable, for example controlled by the control unit, to control the temperature of provided thermoregulation fluid independently of the operation or the rate of operation of the input and output pumps and independently of operation of the pressure control system.

In some embodiments, the heat control unit is operable independently of the input and output pumps and independently of the pressure control system.

In some embodiments, the heat control unit includes a temperature regulated fluid supply, and a mixing system. The regulated fluid supply can include a hot fluid supply for supplying hot fluid and a cold fluid supply for supplying cold fluid.

The mixing system can be operated by the control unit.

In some embodiments, the mixing system is operable to circulate thermoregulation fluid from the temperature regulated fluid supply to the temperature regulated fluid supply without passing through a thermoregulation pack.

In some embodiments, the mixing system is operable to circulate thermoregulation fluid from the hot tank to the hot tank without passing through a thermoregulation tank and preferably without passing to or through the cold tank.

In some embodiments, the mixing system is operable to circulate thermoregulation fluid from the cold tank to the cold tank without passing through a thermoregulation tank and preferably without passing to or through the hot tank.

Preferably, the heat control unit includes a hot fluid supply and a cold fluid supply and a mixing system for mixing hot fluid from the hot fluid supply with cold fluid from the cold fluid supply to provide thermoregulation fluid at a predetermined thermoregulation temperature.

In some embodiments, the heat control unit includes a hot fluid supply for supplying hot fluid and a cold fluid supply for supplying cold fluid; and the control unit is operable selectively to operate the output pump to pump out through the output port thermoregulation fluid from the hot fluid supply, thermoregulation fluid from the cold fluid supply, or thermoregulation fluid including a mix of thermoregulation fluid from the hot and cold fluid supplies; and the control unit is operably selectively to operate the input pump to pump thermoregulation fluid in through the input port to the cold fluid supply, to the hot fluid supply, or to both the cold and hot fluid supplies.

Preferably, the mixing system includes a return feed for returned or recycled thermoregulation fluid, the mixing system being operable to mix fluid from the regulated fluid supply, such as hot fluid from the hot fluid supply and cold fluid from the cold fluid supply, with returned or recycled thermoregulation fluid, to provide thermoregulation fluid at a predetermined thermoregulation temperature.

In some embodiments, the return feed can be selectively coupled, for example by a first recycling or pack bypass conduit, to an output of the mixing system whereby to recycle or bypass thermoregulation fluid from, for example directly from, the mixing system to the return feed without passing through a thermoregulation pack. This can advantageously be used to bring a volume of thermoregulation fluid to a desired temperature before it is passed to a pack, preventing a patient being exposed to overly hot or overly cold temperatures.

In some embodiments, an input junction of a regulated fluid supply unit can be selectively coupled, for example by a recycling conduit, to an output junction of the regulated fluid supply unit, whereby to recycle thermoregulation fluid for example without passing through the regulated fluid supply.

In embodiments, the regulated fluid supply unit is a unit for outputting fluid from and inputting fluid to the regulated fluid supply and preferably includes the regulated fluid supply.

In some embodiments, an output junction of the regulated fluid supply unit, preferably of the hot fluid supply, can be selectively coupled, for example by a pack bypass conduit, to an input junction of the regulated fluid supply unit, preferably also of the hot fluid supply, whereby to bypass thermoregulation fluid from the said output junction of the regulated fluid supply unit to the said input junction of the regulated fluid supply unit without passing through a thermoregulation pack.

In some embodiments, the pack bypass conduit can serve as a recycling conduit to recycle thermoregulation fluid from a thermoregulation pack to a thermoregulation pack without passing through the regulated fluid supply.

A pack bypass pump can be provided coupled to the pack bypass conduit to pump thermoregulation fluid from an output junction of the regulated fluid supply unit to an input junction of the regulated fluid supply unit.

In some embodiments, a second recycling conduit is provided which can be selectively activated to recycle thermoregulation fluid from a thermoregulation pack to a thermoregulation pack without passing through the regulated fluid supply. A recycling pump can be coupled to the second recycling conduit to selectively pump thermoregulation fluid in a circulation that avoids the regulated fluid supply.

In some embodiments, the pack bypass pump can serve as the recycling pump for pumping thermoregulation fluid in a circulation that avoids the regulated fluid supply and the pack bypass conduit can serve as the second recycling conduit.

The thermoregulation system preferably includes a mixing system bypass conduit which can be selectively activated to cause thermoregulation fluid entering the input port to be directed to the output port without passing through the mixing system. This can advantageously be used to isolate fluid in a pack from the mixing system, for example when the mixing system is being operated to bring a volume of fluid to a desired temperature.

Selective coupling and selective activation of a conduit can be provided by a valve which is operable, for example under control of the control unit, to selectively direct incoming fluid to one or more of a plurality of output conduits.

Selective coupling can be provided by a conduit coupled to a selectively operable pump which is configured to prevent thermoregulation fluid flow through that conduit in a non-operating state and to pump thermoregulation fluid through that conduit in an operating state. Similarly, selective activation of a conduit can be provided by providing coupled to that conduit a selectively operable pump which is configured to prevent thermoregulation fluid flow through that conduit in a non-operating state and to pump thermoregulation fluid through that conduit in an operating state.

Couplings between components configured to direct or convey thermoregulation fluid can be provided by conduits configured to convey thermoregulation fluid therebetween.

A mixing system bypass pump is preferably coupled to the mixing system bypass conduit to pump or drive fluid through a thermoregulation pack and along the mixing system bypass conduit. The mixing system bypass pump can be reversible.

In some embodiments the pack bypass conduit and/or the second recycling conduit, which may be the same conduit, can be selectively activated owing to the pack bypass pump and/or recycling pump, which may be the same pump, being a selectively operable pump which is configured to prevent thermoregulation fluid flow through the respective conduit in a non-operating state and to pump thermoregulation fluid through the respective conduit in an operating state. Furthermore, in these embodiments, the mixing system bypass conduit can be selectively activated by the mixing system bypass pump being a selectively operable pump which is configured to prevent thermoregulation fluid flow through the mixing system bypass conduit in a non-operating state and to pump thermoregulation fluid through the mixing system bypass conduit in an operating state. In these embodiments, the input and output pumps can be selectively operable pumps configured to prevent flow in a non-operating state, but other selective couplings or selective activations in the thermoregulation supply or supply system are provided by valves.

The input and/or output pump can be provided in the mixing system.

The input pump can be directly coupled to an input of the mixing system.

The output pump can be directly coupled to an output of the mixing system.

An output of the input pump can be coupled, for example selectively coupled, to the heat control unit or to a regulated fluid supply unit to return thermoregulation fluid thereto.

A recycling conduit can be selectively coupled between an output of the input pump and an input of the output pump example in order to circulate thermoregulation fluid without passing it to the regulated fluid supply unit or in some embodiments without passing it to the heat control unit. This can be advantageous for example for maintaining thermoregulation fluid at a current temperature, or for circulating thermoregulation fluid within the thermoregulation system to bring it to a desired temperature before sending it to a pack.

The input pump and/or the output pump and/or the pack bypass pump can be reversible.

Where components are described as coupled, then unless they are described as directly coupled, other components may be coupled between them.

The thermoregulation system is preferably operated so that the pressurising element applies a pressure less than a pressure applied by thermoregulation fluid in the pack.

In some embodiments, the input, output and bypass pumps are connected to a variable valve configuration manifold which also means the system can send from one of two tanks (cold and hot) or a blend of both using one pump and then return to the pack via the second pump to the same or different tank via this manifold or indeed bypassing both tanks and simply circulate around the pack only using the third pump.

Preferably, each of the pumps of the thermoregulation system is selectively operable and independently controllable by the control unit, for example, the control unit can independently control a pumping rate of each pump.

According to an aspect of the invention, there is provided a personal thermoregulation pack for the above thermoregulation system, the pack including:
a thermoregulation zone for thermoregulation fluid;
a pressurising element for applying pressure to the thermoregulation zone to press the thermoregulation zone against a patient;
one or more thermoregulation fluid inlets coupled to the thermoregulation zone; and
one or more thermoregulation fluid outlets coupled to the thermoregulation zone;
wherein the pack includes no liquid restricted return outlet.

According to an aspect of the invention, there is provided a kit including a thermoregulation system as above and at least one personal interface pack for coupling to the system.

Preferably, the kit includes a plurality of personal interface packs.

Preferably, the or each pack includes:
an input for coupling to the output port of the thermoregulation system;
an output for coupling to the input port of the thermoregulation system;
a thermoregulation zone for conveying thermoregulation fluid from the input to the output; and
a pressurising element for applying pressure to the thermoregulation zone to press the thermoregulation zone against a patient.

In embodiments, the or each pack is tubular and the thermoregulation zone is radially inward with respect to the pressurising element.

In embodiments, the or each pack is for being placed entirely around a user's limb, for example is a cuff.

In embodiments, the pressurising element of the or each pack extends fully over the thermoregulation zone.

The thermoregulation zone can be or include a thermal chamber.

According to an aspect of the invention, there is provided a control unit for operating a thermoregulation system, the thermoregulation system including an output port for coupling to an input of a personal thermoregulation pack, an input port for coupling to an output of a personal thermoregulation pack, a thermoregulation fluid supply system for supplying thermoregulation fluid to the output port, an output pump for pumping thermoregulation fluid out through the output port, an input pump for pumping thermoregulation fluid in through the input port; and, a pressure control system for selectively operating a pressurising element of a personal thermoregulation pack to apply a pressure independently of operation of the input and output pumps; the control unit being operable to, operate the output pump to pump thermoregulation fluid out through the output port, operate the input pump to pump thermoregulation fluid in through the input port and operate the pressure control system to cause a pressurising element of a personal thermoregulation pack to apply a pressure to a personal thermoregulation pack.

According to an aspect of the invention, there is provided a method of operating a thermoregulation system, the thermoregulation system including:
an output port for coupling to an input of a personal thermoregulation pack;
an input port for coupling to an output of a personal thermoregulation pack;
a thermoregulation fluid supply system for supplying thermoregulation fluid to the output port;
an output pump for pumping thermoregulation fluid out through the output port;
an input pump for pumping thermoregulation fluid in through the input port; and
a pressure control system for selectively operating a pressurising element of a personal thermoregulation pack to apply a pressure independently of operation of the input and output pumps; the method including:
operating the output pump to pump thermoregulation fluid out through the output port;
operating the input pump to pump thermoregulation fluid in through the input port; and
operating the pressure control system to cause a pressurising element of a personal thermoregulation pack to apply a pressure to a personal thermoregulation pack.

According to an aspect of the invention, there is provided a method of operating a thermoregulation system, the thermoregulation system including:
an output port for coupling to an input of a personal thermoregulation pack;
an input port for coupling to an output of a personal thermoregulation pack;
a thermoregulation supply for supplying thermoregulation fluid to the output port;
an output pump for pumping thermoregulation fluid out through the output port;
an input pump for pumping thermoregulation fluid in through the input port; and
a pressure control system for selectively operating a pressurising element of a personal thermoregulation pack to apply a pressure independently of operation of the input and output pumps; the method including:
operating the output pump to pump thermoregulation fluid out through the output port;
operating the input pump to pump thermoregulation fluid in through the input port; and
operating the pressure control system to cause a pressurising element of a personal thermoregulation pack to apply a pressure to a personal thermoregulation pack.

The thermoregulation system of the method can include any of the features of the thermoregulation system described above.

Preferably, the output pump and optionally the input pump is operated to provide a desired volume of thermoregulation fluid to a pack before the pressure control system is operated.

The method can include operating the output pump and the input pump at the same rate to maintain a fixed volume of thermoregulation fluid in a thermoregulation pack.

The method can include varying the rate of operation of the output pump and input pump equally, and preferably simultaneously, to maintain a fixed volume or a fixed rate of change of volume of thermoregulation fluid in a thermoregulation pack.

The method can include operating the output pump at a higher rate than the input pump to increase a volume of thermoregulation fluid in a thermoregulation pack.

The method can include operating the output pump at a lower rate than the input pump to decrease a volume of thermoregulation fluid in a thermoregulation pack.

The method can include operating the pressure control system to vary a pressure applied by a pressurising element or chamber of a personal interface pack.

The method preferably includes operating a heat control unit, such as the heat control unit above, to provide thermoregulation fluid at a predetermined thermoregulation temperature.

The control unit can be operable to perform any or each of the above method steps.

Embodiments include software databases for how to prime and run a pack for optimal performance and has a connected effect to efficiency of therapy. Based upon the efficiency of the pack, a unique and special database and algorithm can be provided that takes:

$$\{ Air in outer chamber + Pressure of air in outer chamber + Temperature of air in outer chamber + Volume of coolant in pack = Surface area of coolant in contact of human + current average temperature across pack + /= define flow rate of coolant + real time temperature + pressure target + pack database table \}$$

According to an aspect of the invention, there is provided a program for performing any one or more of the above methods when executed on a computing device, such as a control unit as described herein.

According to an aspect of the invention, there is provided a computing device programmed with the above program or otherwise configured to perform the steps of any one or more of the above methods. The computing device can be a control unit of a thermoregulation system, for example the control unit described herein. Where the control unit is described herein as being operable to perform a particular function, it is to be appreciated that it can be programmed or otherwise configured to perform that function.

It is to be appreciated that certain embodiments of the invention as discussed herein may be incorporated as code (e.g., a software algorithm or program) residing in firmware and/or on computer useable medium having control logic for enabling execution on a computer system having a computer processor. Such a computer system typically includes memory storage configured to provide output from execution of the code which configures a processor in accordance with the execution. The code can be arranged as firmware or software, and can be organized as a set of modules such as discrete code modules, function calls, procedure calls or objects in an object-oriented programming environment. If implemented using modules, the code can comprise a single module or a plurality of modules that operate in cooperation with one another.

### BRIEF DESCRIPTION OF THE DRAWINGS

Embodiments of the invention are described below, by way of example only, with reference to the accompanying drawings, in which:

FIG. 1 is a schematic diagram of a thermoregulation assembly in accordance with an embodiment of the invention;

FIG. 2 is a schematic side view of a thermoregulation pack in accordance with the embodiment of FIG. 1;

FIG. 3 is a cross-sectional view of thermoregulation pack of FIG. 2

FIG. 4 is a schematic diagram of the thermoregulation assembly of the embodiment of FIGS. 1 to 3 showing a pack circulation operation;

FIG. 5 is a schematic diagram of the thermoregulation assembly of the embodiment of FIGS. 1 to 4 showing an internal circulation operation;

FIG. 6 is a schematic diagram of the thermoregulation assembly of the embodiment of FIGS. 1 to 5 showing pack and internal circulation operations simultaneously;

FIG. 7 is a schematic diagram of the thermoregulation assembly of the embodiment of FIGS. 1 to 6 showing a supply operation;

FIG. 8 is a schematic diagram of a thermoregulation assembly according to an embodiment of the invention;

FIG. 9 shows an isometric image of an alternative pack according to an embodiment of the invention;

FIG. 10 shows various cross-sections of the pack of FIG. 9;

FIG. 11 is a schematic diagram of a thermoregulation assembly in accordance with an embodiment of the invention;

FIG. 12 is a schematic diagram of the thermoregulation assembly of the embodiment of FIG. 11 showing a pack circulation operation;

FIG. 13 is a schematic diagram of the thermoregulation assembly of the embodiment of FIGS. 11 to 12 showing an internal circulation operation;

FIG. 14 is a schematic diagram of the thermoregulation assembly of the embodiment of FIGS. 11 to 13 showing pack and internal circulation operations simultaneously;

FIG. 15 is a schematic diagram of the thermoregulation assembly of the embodiment of FIGS. 11 to 14 showing a supply operation;

FIG. 16 is a schematic diagram of a thermoregulation assembly according to an embodiment of the invention; and

FIG. 17 is a schematic diagram of the thermoregulation assembly of the embodiment of FIGS. 11 to 15 showing a hot tank internal circulation operation.

### DETAILED DESCRIPTION OF PREFERRED EMBODIMENTS

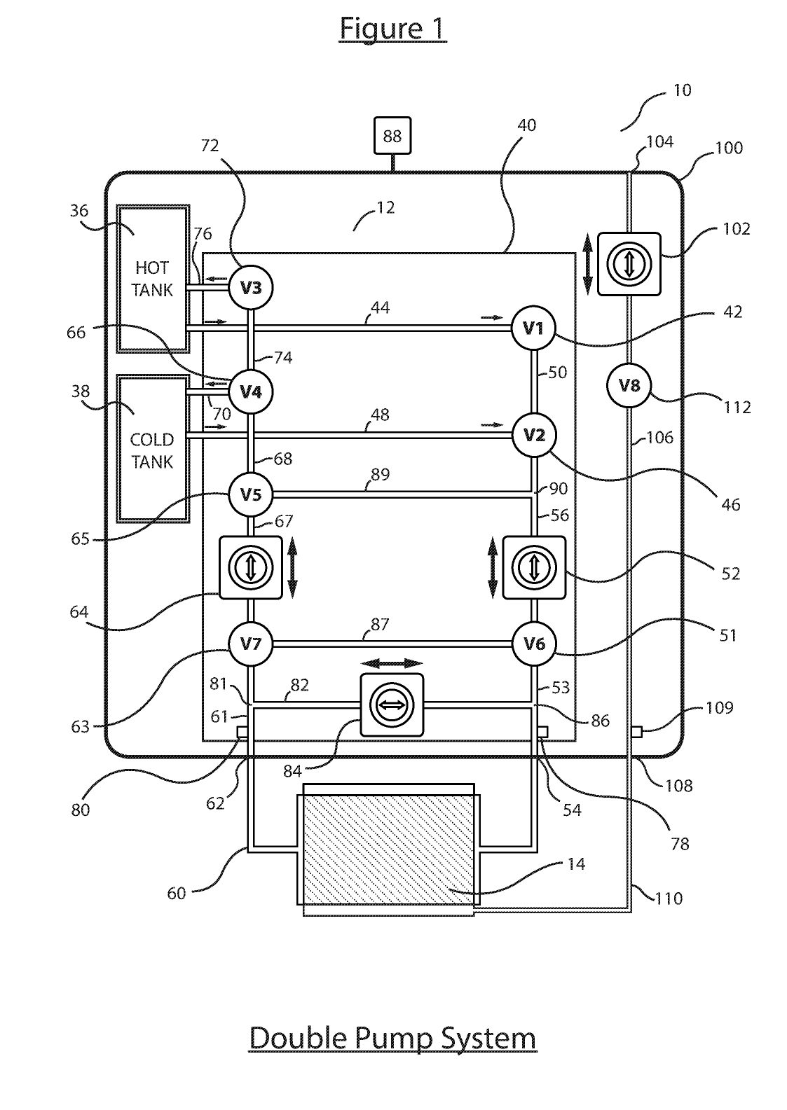

As can be seen from FIG. 1, a thermoregulation assembly 10 includes a thermoregulation fluid supply system 12 and a thermoregulation pack 14.

The thermoregulation pack 14 is an interface pack for being placed around or against a part of a patient's body in order to apply a thermal treatment to alleviate or cure physical injuries or disorders.

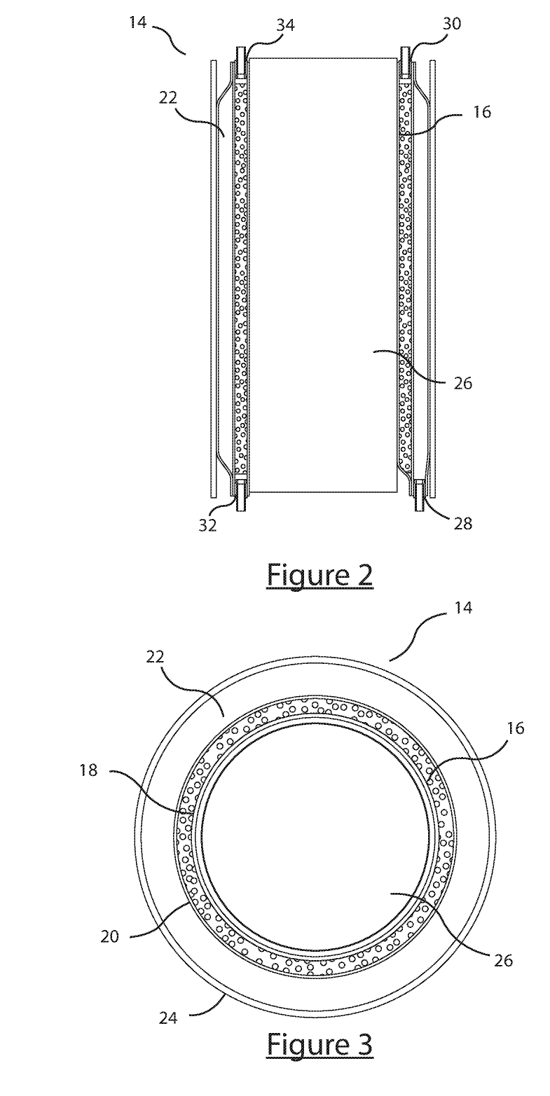

An example of a thermoregulation pack 14 is shown in FIGS. 2 and 3. In this example, the thermoregulation pack is a cuff for example for going around a patient's limb. However, other designs and configurations of thermoregulation pack can be used. WO2013/190333, WO2013/190337, and WO2013/190336, each of which is incorporated herein by reference in its entirety, describe types of interface pack, any of which can be used in the thermoregulation assembly of embodiments of the present invention. However, in preferred embodiments, the thermoregulation pack 14 has a pressurising element which is separate from a thermal chamber.

As can be seen from FIGS. 2 and 3, in this embodiment the pack 14 is a cylindrical cuff.

In this embodiment, the cuff 14 includes a thermal chamber 16 which is defined by the cylindrical space between a cylindrical inner wall 18 and a cylindrical separator wall 20. The distance between the cylindrical inner wall 18 and the cylindrical separator wall 20 is a thickness of the thermal chamber 16. A pressure chamber 22 is defined by the cylindrical space between separator wall 20 and outer wall 24, such that pressure chamber 22 completely surrounds thermal chamber 16 and is concentric with it. The hollow space defined by tubular inner wall 18 is the space into which a body part 26 is placed prior to treatment and can be considered to be a treatment zone. The cylindrical inner wall, to the extent that it forms a boundary between the treatment zone and the thermal chamber, forms a treatment region through which thermal treatment is applied to a user's limb by the thermal chamber.

In this embodiment, the thermal chamber 16 is filled with an open celled foam.

FIG. 2 shows a side view of tubular cuff 14. Inner wall 18, separator wall 20 and outer wall 24 are formed from three tubes and these are bonded together at either end of the cuff 14 in order to terminate the thermal chamber 16 and the pressure chamber 22. The tubes may be hollow to enable fluid to be conveyed along them or alternatively may simply be formed by flexible solid material to function solely as a wall.

The thermal chamber 16 and the pressure chamber 22 are supplied with cooling/heating thermoregulation fluid and pressure fluid respectively through the ends of tubular cuff 14. The pressure fluid feed tube is connected to the pressure chamber 22 via a tubing port 28. A thermoregulation fluid feed is connected to the thermal chamber 16 via tubing port 30. A thermoregulation fluid return tube is connected to the thermal chamber 16 via tubing port 32. A restricted liquid return tube can be coupled to the thermal chamber 16 via tubing port 34.

Further details of the configuration of the thermoregulation pack 14 can be found in WO2013/190336 and WO2014/188213, which are incorporated herein by reference in their entirety.

For reasons that will be evident from the description below, it is not necessary in all embodiments to provide tubing ports for a restricted liquid return. In addition, it is not necessary to provide a pressure chamber 22; other pressurising elements can be used. Furthermore, any of the other modifications described in WO2013/190336 can be applied to the pack 14 described herein, or other designs of pack can be used.

Returning to FIG. 1, the configuration of the thermoregulation fluid supply system 12 is described.

The thermoregulation fluid supply system 12 includes a hot fluid supply 36. In this embodiment the hot fluid supply is a hot tank of thermoregulation fluid. However, in other embodiments, the hot fluid supply 36 can be a port for receiving hot thermoregulation fluid from an external source. Typically, the thermoregulation fluid will be liquid. In the described embodiments the thermoregulation fluid is water, but other fluids especially incompressible liquids can be used in some embodiments.

The thermoregulation fluid supply system 12 also includes a cold fluid supply, which in this embodiment is a cold tank 38. However, in other embodiments, the cold fluid supply can be a port for coupling to an external source of cold thermoregulation fluid.

The hot and cold fluid supplies together form a temperature regulated fluid supply. The hot tank 36 includes a heater and a temperature sensor and a feedback loop between the temperature sensor and the heater in order to maintain thermoregulation fluid in the hot tank at a predetermined temperature. Similarly, the cold tank 38 can include a cooler and a temperature sensor and a feedback loop between the temperature sensor and the cooler in order to maintain thermoregulation fluid in the cold tank at a predetermined temperature.

In other embodiments of the invention, the hot and cold tanks can be replaced by a single thermoregulation fluid supply which supplies fluid at a desired temperature. The thermoregulation fluid supply can include a single tank with a heater/cooler and a temperature sensor and can be operable to raise or lower the temperature of the thermoregulation fluid in the tank to a desired temperature.

The thermoregulation fluid supply system 12 includes a mixing system, in this case in a manifold 40, for mixing hot thermoregulation fluid from the hot tank with cold thermoregulation fluid from the cold tank and/or for mixing thermoregulation fluid from the regulated fluid supply with thermoregulation fluid from the pack 14.

The mixing system and manifold include a number of valves. Discussed below are two port valves and three port valves. Two port valves can be selectively positioned in an open or closed position. In the open position, the valve allows fluid to pass through it. In the closed position, the valve prevents the flow of fluid. The valves can be transitioned from the open to the closed state or vice versa rapidly in response to an electronic control signal. In other embodiments, the two port valves described below can be selectively placed into intermediate states, for example 50% open or 75% open, in order to allow different levels of flow through the valve. However, a valve which can be rapidly transitioned between the open and closed position by an electronic control signal is preferred since the effect of a partially open state can be obtained by rapidly opening and closing the valve. Three port valves have either two input ports and one output port or two output ports and one input port. In some cases, where a port is not specified as input or output, it is because the role of the port can be interchanged during operation.

A three port valve with two input ports and one output port can be selectively positioned in ‘input 1’, ‘input 2’, ‘input both’ or ‘closed’ states. In the ‘input 1’ state, fluid from the first input port is allowed to flow to the output port, but fluid from the second input port is blocked. In the ‘input 2’ state, fluid from the second input port is allowed to flow to the output port, but fluid from the first input port is blocked. In the ‘input both’ state, fluid from both the first and second input ports is allowed to flow to the output port equally. In the ‘closed’ state, fluid is prevented from passing through the valve.

A three port valve with two output ports and one input port can be selectively positioned in ‘output 1’, ‘output 2’, ‘output both’ or ‘closed’ states. In the ‘output 1’ state, fluid from the input port is allowed to flow to the first output port but not the second output port. In the ‘output 2’ state, fluid from the input port is allowed to flow to the second output port, but not the first output port. In the ‘output both’ state, fluid from both the input port is allowed to flow to both the first and second output ports equally. In the ‘closed’ state, fluid is prevented from passing through the valve.

As for the two port valves, the three port valves can be transitioned between the four states rapidly in response to an electronic control signal, thereby effectively being able to produce combinations of the positions. However, in other embodiments, the three port valves can be operable to be positioned part way between two positions, for example to allow a 25% input from the first input and a 75% input from the second input.

The manifold 40 includes a two port hot tank flow valve 42, an input of which is coupled in fluid communication with the hot tank 36 via a conduit 44.

The hot tank flow valve 42 can be selectively positioned in an open or closed state.

The cold tank 38 is coupled in fluid communication with a cold tank flow valve 46 by a conduit 48. The hot tank flow valve 42 and the cold tank flow valve 46 are coupled in fluid communication by a conduit 50. The cold tank flow valve 46 is a three-port valve, with a first input coupled to the hot tank flow valve 42 and a second input coupled to the cold tank 38.

An output of the cold tank flow valve 46 is coupled in fluid communication via a feed or dosing pump 52 to a first port of a flow circulation valve 51 by a conduit 56. The flow circulation valve is a three port valve. A second port of the flow circulation valve 51 is coupled in fluid communication to an output port 54 of the fluid supply system 12 by a conduit 53.

In use, a tube 58 couples the output port 54 of the supply system 12 to a tubing port 30 on the pack 14.

The tubing port 30 is arranged so that in normal operation of the cuff 14 it is no lower than tubing port 32 in order that gravity facilitates the movement and return of fluid within the pack 14.

In use, tubing port 32 is coupled by a tube 60 to an input port 62 of the fluid supply system 12.

The input port 62 is coupled in fluid communication to a first port of a three port return circulation valve 63 by a conduit 61.

A second port of the return circulation valve 63 is coupled in fluid communication via a return pump 64 with a first port of a three port tank circulation valve 65 by a conduit 67.

A second port of the tank circulation valve 65 is coupled in fluid communication to a first input of a three port cold tank return valve 66 by a conduit 68.

A first output of the cold tank return valve 66 is coupled in fluid communication with the cold tank 38 by a conduit 70.

A second output of the cold tank return valve 66 is coupled in fluid communication with a two port hot tank return valve 72 via conduit 74. Hot tank return valve 72 is also coupled in fluid communication with the hot tank 36 via a conduit 76.

The system can include an inline heater, for example coupled to the conduit 76, for instantly heating fluid passing to the hot tank 36. The inline heater can be driven by fluid leaving the hot tank 36 by conduit 44 to ensure all fluid passing to the hot tank is as hot or hotter than fluid leaving the hot tank, thereby ensuring the hot tank maintains a fixed temperature.

Coupled to the conduit 53 between the flow circulation valve 51 and the output port 54 is a solid state safety cell 78. The solid state safety cell 78 includes a pressure sensor and a temperature sensor for measuring the temperature and pressure of thermoregulation fluid leaving the fluid supply system 12.

Coupled to the conduit 61 between the input port 62 and the return circulation valve 63 is a temperature sensor 80 for measuring the temperature of fluid passing through the input port 62.

Between the input port 62 and the return circulation valve 63, in particular in between temperature sensor 80 and the return circulation valve, is a junction 81 which couples a conduit 82 to the conduit 61. The conduit 82 can be considered to be a mixing system bypass conduit and couples the junction 81 to a junction 86 via a circulation or mixing system bypass pump 84. The junction 86 is coupled to the conduit 53 between the flow circulation valve 51 and the output port 54, in particular between the flow circulation valve 51 and the solid state safety cell 78.

The pumps 52, 64 and 84 can be reversible so as to be able selectively to pump fluid in either direction.

A third port of the return circulation valve 63 is coupled to a third port of the flow circulation valve 51 via a conduit 87 which can be considered a pack bypass conduit. A third port of the tank circulation valve 65 is coupled via a conduit 89 to conduit 56 at a junction 90 between the cold tank flow valve and the dosing or feed pump 52. The conduit 89 can be considered to be a recycling conduit.

There are no reservoirs in a fluid path from the feed pump 52 to the output port 54 or in a fluid path from the input port 62 to the return pump 64. This can mean that the pumps are able effectively to pump fluid into and out of a pack 14.

The thermoregulation fluid supply system 12 includes a control unit 88. Each of the temperature and pressure sensors described above is coupled to pass signals to the control unit 88. Furthermore, each of the heaters and coolers and pumps and valves described above is controllably coupled to the control unit 88 so that the control unit can operate them.

In the present embodiment, the mixing system can be considered to include the input and output pumps, the valves 42, 46, 65, 66 and 72, and the conduits 50, 56, 67, 68, 74 and 89. The manifold generally includes more components than just the mixing system. However, the precise extent of the mixing system is not important and could be considered to be any subset of the components of the manifold that serves to mix thermoregulation fluid so as to provide thermoregulation fluid at a desired temperature.

The hot and cold tanks together with the conduits 44, 48, 70 and 76, and the valves 42, 46, 66 and 72 can be considered to provide a regulated fluid supply unit, the valves 66 and 72 providing input junctions thereto and the valves 42 and 46 providing output junctions therefrom.

The thermoregulation fluid supply system, in particular the thermoregulation fluid pumps, are configured to supply or pump thermoregulation fluid at a fluid flow rate in the range from 200 ml/min to 5000 ml/min so that the thermoregulation fluid supply system can supply thermoregulation fluid to, or draw thermoregulation fluid from, the pack at this rate. However, this range can in other embodiments be different depending on the requirements of the assembly. In some embodiments, the range can be from 500 ml/min to 2000 ml/min. In some embodiments, the average fluid flow rate of thermoregulation fluid is about 1000 ml/min.

The thermoregulation assembly 10 also includes a pressure control system including a pressure fluid supply system 100. The thermoregulation fluid supply system and the pressure control system can be said to provide a thermoregulation system. The pressure fluid supply system includes a pressure fluid pump 102 which can pump pressure fluid, typically air, from a pressure fluid source 104, typically the atmosphere, via a conduit 106 to a pressure fluid outlet 108. The pressure fluid outlet 108 can in use be coupled by a tube 110 to tubing port 28 of the pack 14. The pressure fluid supply system 100 can also include a pressure fluid valve 112.

The pressure fluid supply system also includes a pressure fluid sensor 109 for measuring the pressure of the pressure fluid in an attached pack and for measuring a volume of pressure fluid in an attached pack.

The pressure fluid pump 102 is reversible. The pressure fluid pump 102 and the pressure fluid valve are both coupled to the control unit 88 to be controllable thereby. The control unit is configured to operate the pressure fluid supply system so that the pressure of the pressure fluid in the pressure chamber is always at a lower pressure than the pressure of thermoregulation fluid in the thermal chamber. This can prevent the pressure chamber from causing blockages in the thermal chamber.

The thermoregulation assembly 10 can also include a port for receiving fluid from a liquid restricted return. However, since this is not necessarily in all embodiments, it is not described in detail herein.

Further details of the pressure fluid supply system and the liquid restricted return port can be found for example in WO2013/190336.

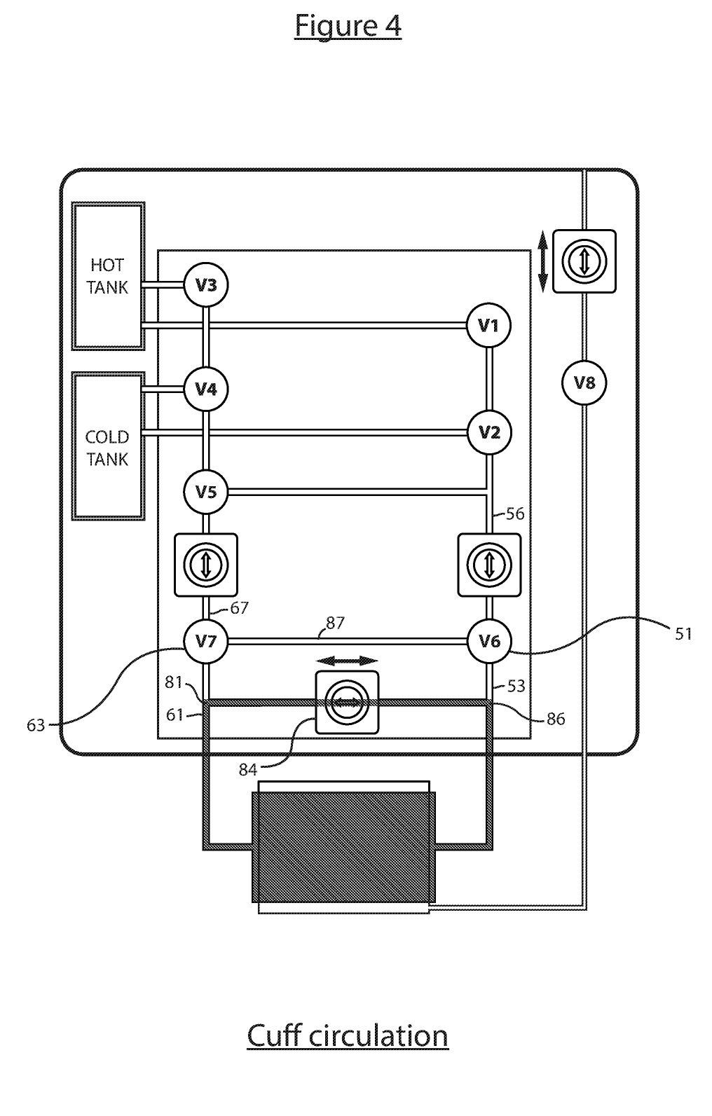

During operation, it is possible for thermoregulation fluid to be circulated around the pack 14 without passing into the mixing system or through the hot and cold tanks of the fluid supply system 12. This is shown in FIG. 4. In order to do this, the return circulation valve 63 can be considered to have an input from conduit 61, and outputs to conduits 67 and 87. The return circulation valve 63 is operated in a “closed” state in order to block fluid from conduit 61.

Flow circulation valve 51 can be considered to have inputs from conduits 87 and 56 and an output to conduit 53. The flow circulation valve 51 is operated in a “input neither” state in which no fluid is passed to conduit 53.

The circulation or mixing system bypass pump 84 is operated to pump fluid in the direction of junction 86. Fluid, being unable to enter the flow circulation valve 51, passes through output port 54 and into pack 14. At the same time, the pump 84 is drawing fluid from junction 81, creating a negative pressure. As no fluid passes through the return circulation valve 63, this draws fluid from the pack 14 via the input port 62 and into the conduit 82. In this way, fluid is circulated through the pack 14 without passing through heat control apparatus. The circulation is carefully controlled because the pump 84 draws from the pack exactly what it provides to the pack. As there are no reservoirs between the pump 84 and the pack 14 in either direction, the positive pressure on one side of the pump 84 is able to drive fluid into the pack 14 and the negative pressure on the other side of the pump 84 is able to draw fluid out of the pack 14.

In some embodiments, the pump 84 can be operated in the opposite direction, which would be anti-clockwise in FIG. 4. However, since packs 14 typically have a preferred operating direction, this is not usually preferable.

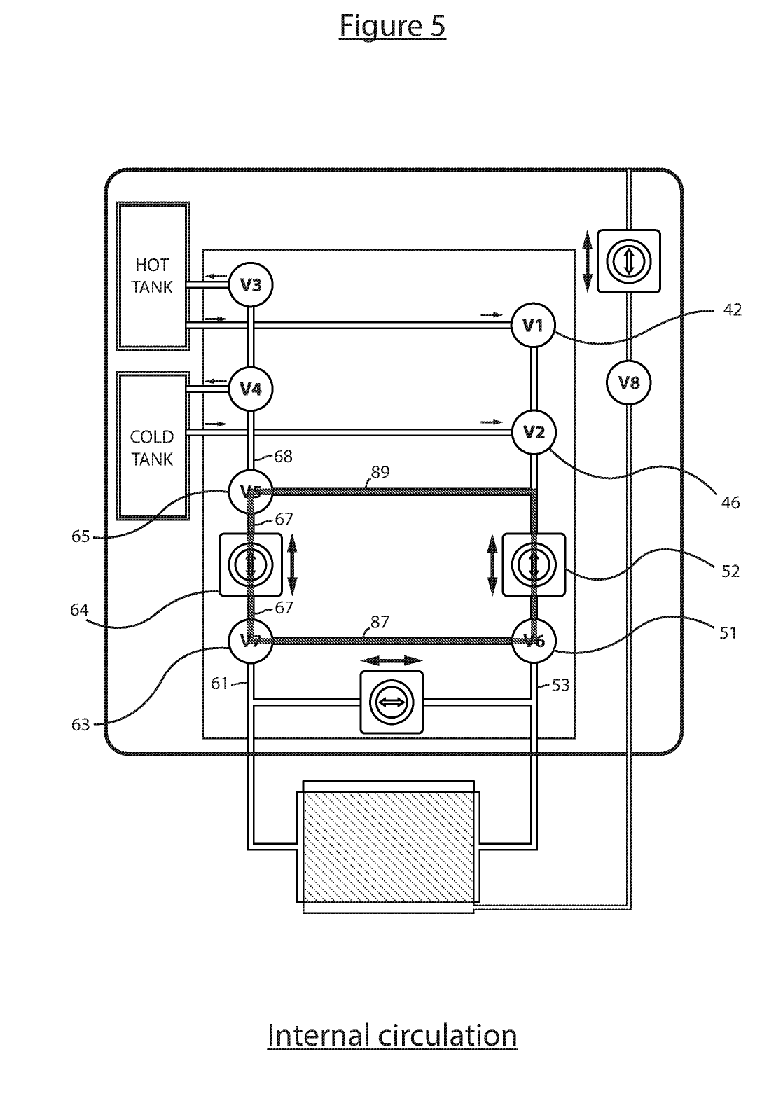

The assembly 10 can also be operated in an internal circulation mode, shown in FIG. 5. In this situation, fluid is circulated within the fluid supply system 12 without passing to the pack 14. As can be seen from FIG. 5, in this situation, the tank circulation valve 65 can be considered to have an input from conduit 67, a first output to conduit 68 and a second output to conduit 89. The return circulation valve 63 can be considered to have a first input from conduit 61, a second input from conduit 87, and an output to conduit 67. The flow circulation valve 51 can be considered to have an input from conduit 56, a first output to conduit 53, and a second output to conduit 87.

The tank circulation valve 65 is operated in a “output 2” mode, meaning that fluid from conduit 67 is passed only to conduit 89. Return circulation valve 63 is operated in a “input 2” mode meaning that only fluid from conduit 87 is passed to conduit 67. The flow circulation valve 51 is operated in a “output 2” mode meaning that fluid from conduit 56 is passed only to conduit 87. The return pump 64 is operated to pump fluid from return circulation valve 63 towards the tank circulation valve 65, and the dosing or feed pump 52 is operated to pump fluid from the tank circulation valve 65 towards the flow circulation valve 51. This causes fluid to continuously flow in a clockwise direction as shown in FIG. 5.

The internal circulation shown in FIG. 5 can be used advantageously to bring thermoregulation fluid to a desired temperature before passing it through the pack 14. In order to do this, the tank circulation valve 65 can rapidly be switched from “output 2” mode to “output 1” mode in order to pass thermoregulation fluid into conduit 68 and, by appropriate operation of the hot tank return valve and/or the cold tank return valve, this thermoregulation fluid can be returned to the hot or cold tanks. In addition, preferably simultaneously with the transition of the tank circulation valve 65 to the “output 1” mode, the hot tank flow valve and/or the cold tank flow valve 46 can be operated in order to allow a desired quantity of hot thermoregulation fluid from the hot tank and/or cold thermoregulation fluid from the cold tank to be drawn towards the dosing or feed pump 52 to join the thermoregulation fluid circulating. The tank circulation valve 65 is rapidly transitioned back to the “output 2” mode, and the cold tank flow valve 46 is transitioned into a “closed” mode. This can be repeated at intervals in order to gradually introduce hot and/or cold thermoregulation fluid to the circulating thermoregulation fluid in order slowly to bring the thermoregulation fluid to a desired temperature.

The internal circulation can be operated in an anti-clockwise manner by reversing the operation of the dosing or feed pump 52 and the return pump 64. However, this is not desirable since it makes it difficult to add hot and/or cold thermoregulation fluid from the hot and/or cold tanks.

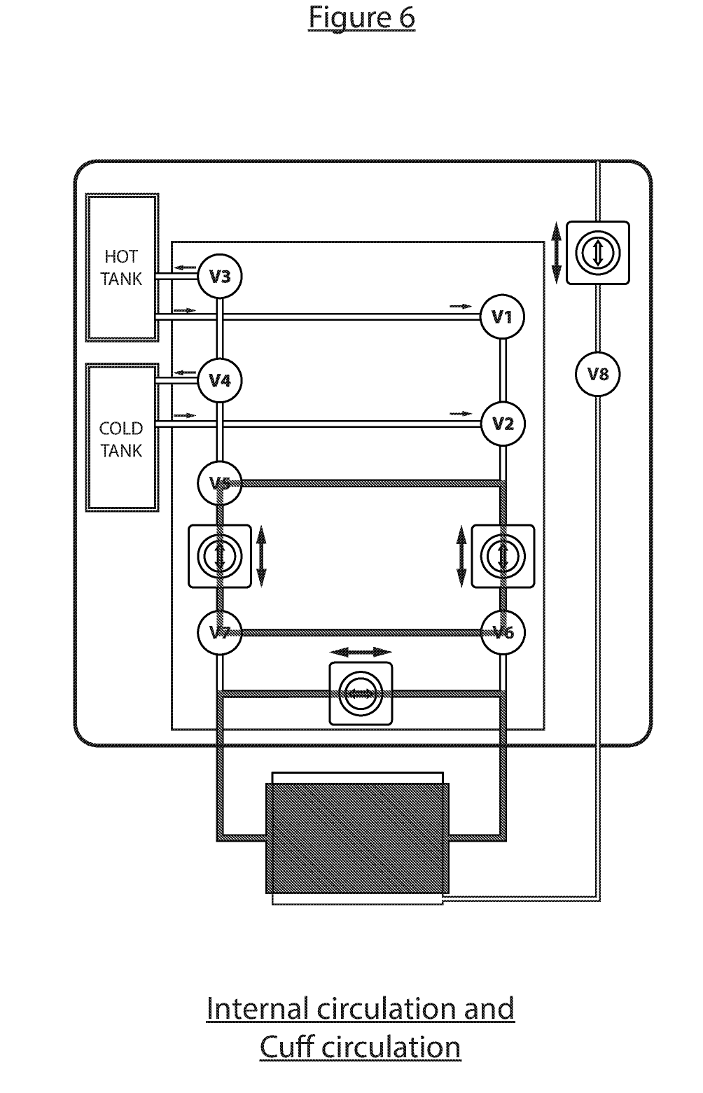

The internal circulation and the pack circulation can be operated simultaneously in order to circulate the existing pack fluid in the pack while further fluid is brought to the correct temperature. This is shown in FIG. 6.

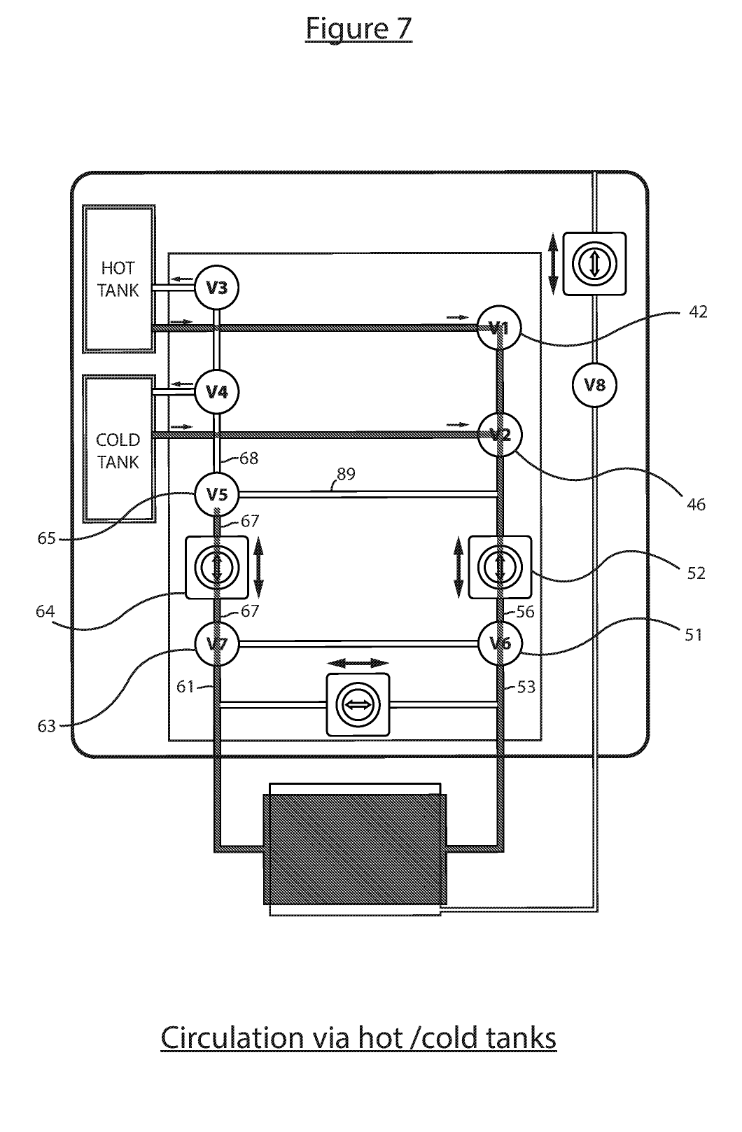

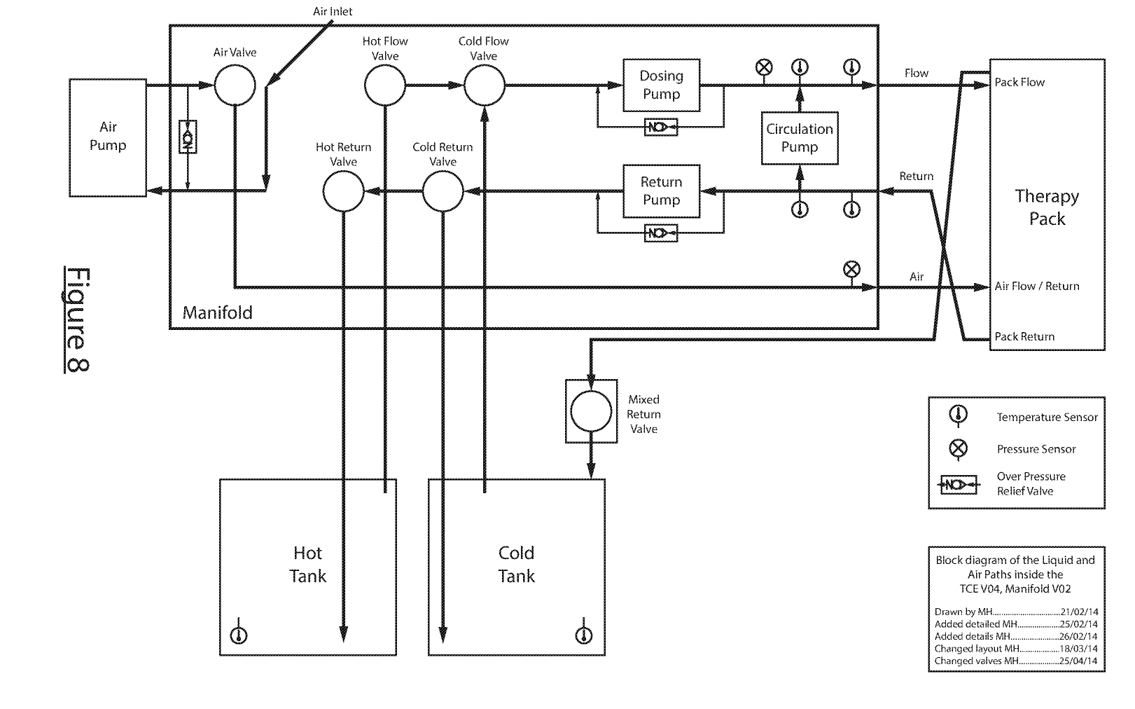

As shown in FIG. 7, the assembly 10 can be operated to provide thermoregulation fluid directly to the pack 14 from the mixing system in particular from the hot and/or cold tanks. In this situation, the flow circulation valve 51 can be operated to allow fluid to pass only from conduit 56 to conduit 53; the return circulation valve can be operated to allow fluid to pass only from conduit 61 to conduit 67; and the tank circulation valve can be operated to allow fluid to pass only from conduit 67 to conduit 68. The hot and cold tank return valves can be operated as appropriate to allow fluid to pass into the hot and/or cold tanks.

The hot tank flow valve 42 and the cold tank flow valve 46 can be operated by the control unit 88 to transition between states in a rapid manner. These can therefore be operated by the control unit rapidly to switch between states in order to provide a desired ratio of hot fluid to cold fluid in order to provide thermoregulation fluid flowing into conduit 56 and then into conduit 53 and into the pack at a desired temperature in accordance with the desired feed parameters.

It is also possible for the tank circulation valve 65, instead of passing fluid from conduit 67 to conduit 68, to pass fluid from conduit 67 to conduit 89, and thereby recycle fluid within the mixing system, where it can optionally be mixed with fluid from the hot and/or cold tanks to adjust the temperature. In this mode of operation, the cold tank flow valve and hot tank flow valves are open for less than in the mode shown in FIG. 7 since, unlike FIG. 7, a large amount of the thermoregulation fluid to be supplied to the pack is recycled fluid from the pack. Preferably, the valves 42 and 46 only allow the addition of fluid from the hot and/or cold tank at intervals. As per the mode described in connection with FIG. 5, when fluid is being added from the hot and/or cold tanks, fluid from conduit 67 can be allowed to return to the hot and/or cold tanks to avoid increasing the circulating volumes.

In both this mode of operation and the mode of operation shown in FIG. 7, the feed or dosing pump 52 is operated to pump fluid into the pack at a predetermined rate, and the return pump 64 is operated to draw fluid out of the pack at a predetermined rate. As discussed elsewhere, the rates of the feed and return pumps can be equal if it is desired to maintain the existing volume of thermoregulation fluid in the pack, or they can be different if it is desired to change the volume of thermoregulation fluid in the pack.

When a treatment is to be performed, the control unit 88 obtains a desired set of operation or feed parameters. For details of how a desired set of operation or feed parameters can be obtained, reference is made to WO 2013/190336 which is incorporated herein by reference in its entirety. Although WO 2013/190336 describes a different thermoregulation and pressure system, the control system described therein, subject to any changes to account for the differences in the thermoregulation and pressure systems, is applicable to the present embodiment.

The feed or operation parameters include the temperature of the thermoregulation fluid to be supplied to the pack 14, the volume of thermoregulation fluid to be in or supplied to the pack 14, the rate of flow of thermoregulation fluid to the pack and the pressure of the pressure fluid for the pack, to be applied to or to be in the pack. In some embodiments, the feed or operation parameters can include the pressure of thermoregulation fluid for the pack, to be applied to or to be in the pack. However, this is just one example of how to obtain feed or operation parameters; other means of obtaining feed or operation parameters can be used.

In order to obtain thermoregulation fluid at a desired temperature, the control unit can operate the thermoregulation system to bring a volume of fluid to that desired temperature such as shown in FIGS. 5 and 6, or it can mix thermoregulation fluid as it is being fed to the pack as shown in and described for FIG. 7.

While embodiments of the invention as discussed above can bypass the mixing system to keep a pack's thermoregulation fluid circulating around the pack without modification by the mixing system, and/or can bypass the pack to recycle thermoregulation fluid within or to the mixing system, particular advantages of embodiments of the invention are realised when the mixing system is supplying thermoregulation fluid which is circulated through the pack using the feed and return pumps and the below relates to such a mode of operation. This may be after thermoregulation fluid has been brought to temperature using the configurations shown in FIGS. 5 and 6 and/or thermoregulation fluid may be being brought to temperature by the mixing system and circulated to the pack as part of the same cycle.

When thermoregulation fluid is to be supplied to the pack, the control unit operates the pressure fluid pump 102 to provide pressure fluid to the pack in accordance with the operation or feed parameters, and operates the valves and pumps of the thermoregulation fluid supply system 12 to supply thermoregulation fluid in accordance with the operation or feed parameters.

An advantage of having a feed pump 52 and a return pump 64 is that the volume and the flow rate of the thermoregulation fluid in the pack 14 can be accurately controlled. For example, beginning with the pack 14 empty, the control unit 88 can operate the feed pump 52 to pump thermoregulation fluid at a predetermined rate for a predetermined time into the pack 14. This will provide a known volume of fluid into the pack 14. At the end of this initial dosing, the pack 14 will contain a known volume of thermoregulation fluid.

From that point on, the feed pump 52 and return pump 64 can be operated at the same rate as each other. This will ensure that fluid is entering the pack 14 at the same rate as it is leaving the pack 14, thereby keeping the volume of the pack constant. While the prior art could ensure that thermoregulation fluid was entering the pack at a desired rate, in some positions or orientations, thermoregulation fluid could become trapped in the pack, and/or balloon in the pack and not return to the supply system at the same rate. This could cause a change in the volume of thermoregulation fluid in the pack and thereby affect the performance of the pack. Embodiments of the present invention by pumping into the pack at a known rate and pumping out of the pack at a known rate are able to ensure that the volume remains constant. The thermoregulation fluid is not able to become trapped or to balloon into the pack since it is being actively pumped out of the pack and it is not energetically favourable to remain in the pack.

It is mentioned above that it is not necessary in all embodiments to provide a liquid restricted return to the pack 14. This is because in embodiments of the present invention in which fluid is pumped into and out of the pack, all fluid, including air, is forcibly passed through the system and removed; it will not become trapped and interfere with the operation of the pack.

The operation of the feed pump 52 and the return pump 64 can be used to vary the flow rate of the thermoregulation fluid in the pack without varying the volume in the pack since, provided that the feed pump 52 and return pump 64 are being operated at the same rate, the rate can be increased or decreased as desired while maintaining the existing volume of thermoregulation fluid within the pack 14. Varying the flow rate in this manner varies the rate of thermoregulation fluid passing the patient and therefore the rate of energy being provided to or removed from the pack.

Alternatively, if it is desired to change the volume of thermoregulation fluid within the pack 14, the pumps can be operated at different rates. For example, if the feed pump 52 is operated at a higher rate than the return pump 64, the volume of the thermoregulation fluid in the pack 14 will increase at the rate of the difference between pumping rates. Correspondingly if it is desired to reduce the volume of thermoregulation fluid in the pack 14, the return pump 64 can be operated at a higher rate than the feed pump 52, and the volume in the pack 14 will decrease at the rate of the difference between the pumping rates. This can be useful if it is determined that the volume of thermoregulation fluid in the pack is causing too much energy to be transferred to or from the patient, risking burns, or too little energy to be transferred, making the pack inefficient.

While the pumps 52 and 64 are being operated to control the volume and the flow rate of the pack, and the pump 102 is being operated to control the pressure of the pack, the valves of the thermoregulation fluid supply system 12 can be operated as described above to adjust the temperature of the thermoregulation fluid being supplied to the pack.

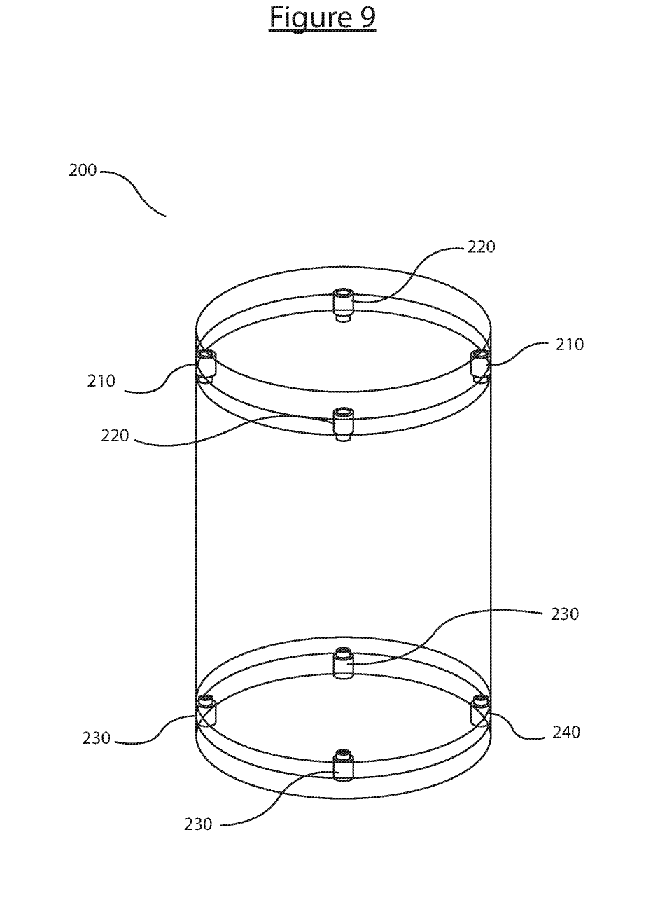

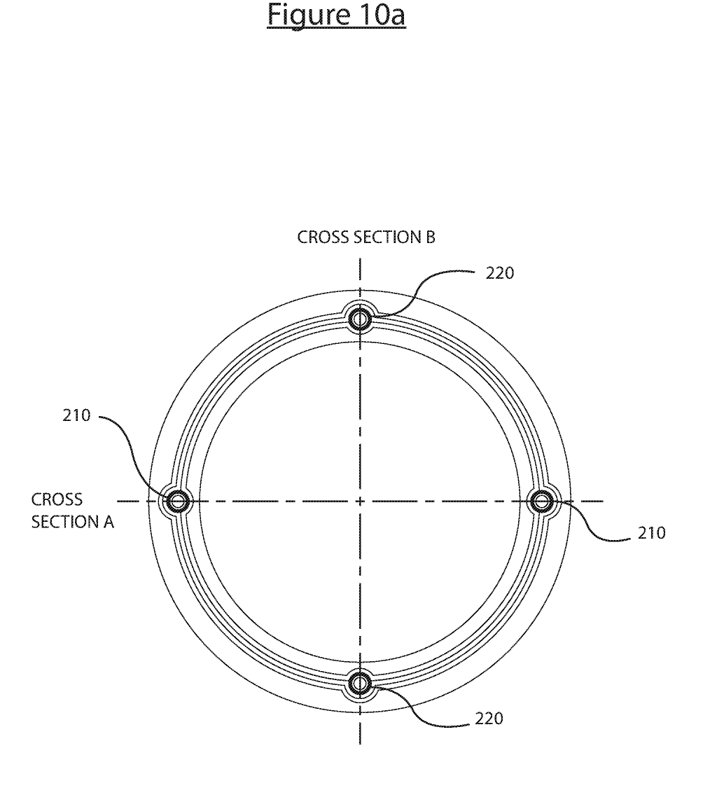

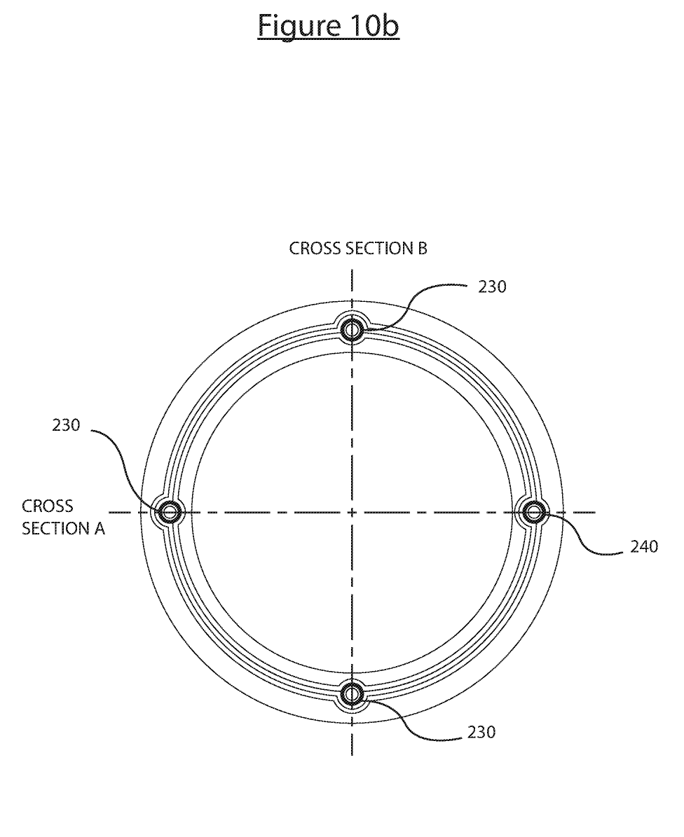

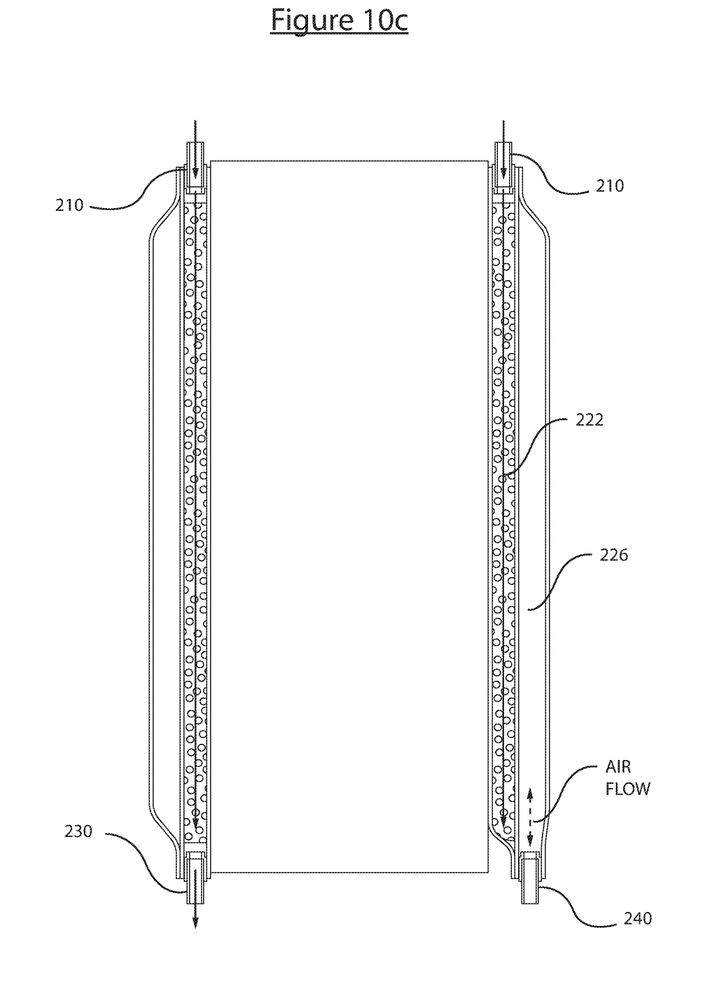

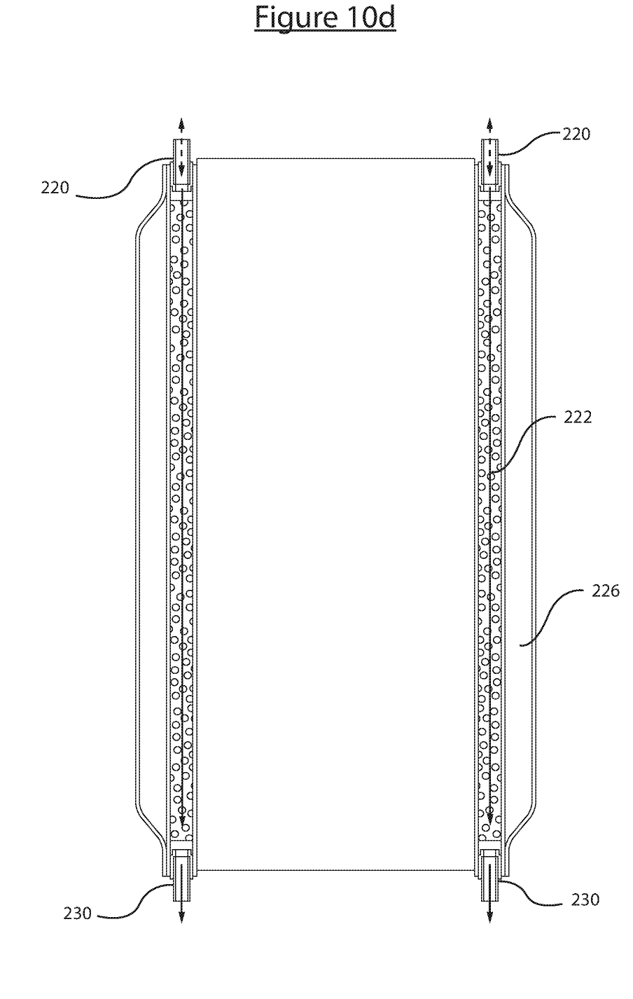

An alternative design of thermoregulation pack, in this case tubular cuff 200, is shown in FIGS. 9 and 10. Cuff 200 has 8 ports in total: 2 liquid feed and 2 restricted liquid return ports at the top, and 3 liquid return and 1 air feed at the bottom. Cuff 200 has thermal chamber 222 which is constructed as described for thermal chamber 16 above and pressure chamber 226 which is constructed as described for pressure chamber 22 above.

In more detail, FIG. 9 shows an isometric view of cuff 200 having two liquid feed ports 210 and two restricted return ports 220 at the top of cuff 200, and three liquid return ports 230 and one air feed port 240 at the bottom of cuff 200.

Cuff 200 is illustrated in more detail in top cross section in FIG. 10A and in bottom cross section in FIG. 10B. FIG. 10C then shows cross section view A and FIG. 10D shows cross section view B through FIGS. 10A and 10B.

In use, heating or cooling liquid is supplied to the cuff via liquid feed ports 210 and flows through the thermal chamber 222 as before, to exit via liquid return ports 230. Restricted return ports 220 function as an exhaust to allow any air to escape from the thermal chamber 222 as above. Pressure fluid is supplied to pressure chamber 226 as per the pack described above.

Although pressure is described above as being provided by a pressurising element in the form of a separate chamber for pressure fluid, other pressurising elements or means for compressing can be used, but is preferably one or more of the following:

(a) a second chamber to push against the thermal chamber when pressurised; or
(b) an outer wall of electroactive material for the thermal chamber connected to an electrical source which contracts when activated and compresses the thermal chamber against the body part.

In embodiments in which the pressurising element does not receive pressure fluid, the operation or feed parameters relating to the pressure do not relate to pressure fluid but relate to other appropriate operation parameters for the pressurising element.

In a particularly preferred embodiment, the means for compressing comprises a second chamber at least partially (and preferably completely) surrounding the thermal chamber and having an inlet and an outlet for a pressurising fluid, whereby putting said fluid under pressure in the second chamber compresses the thermal chamber into the body part.

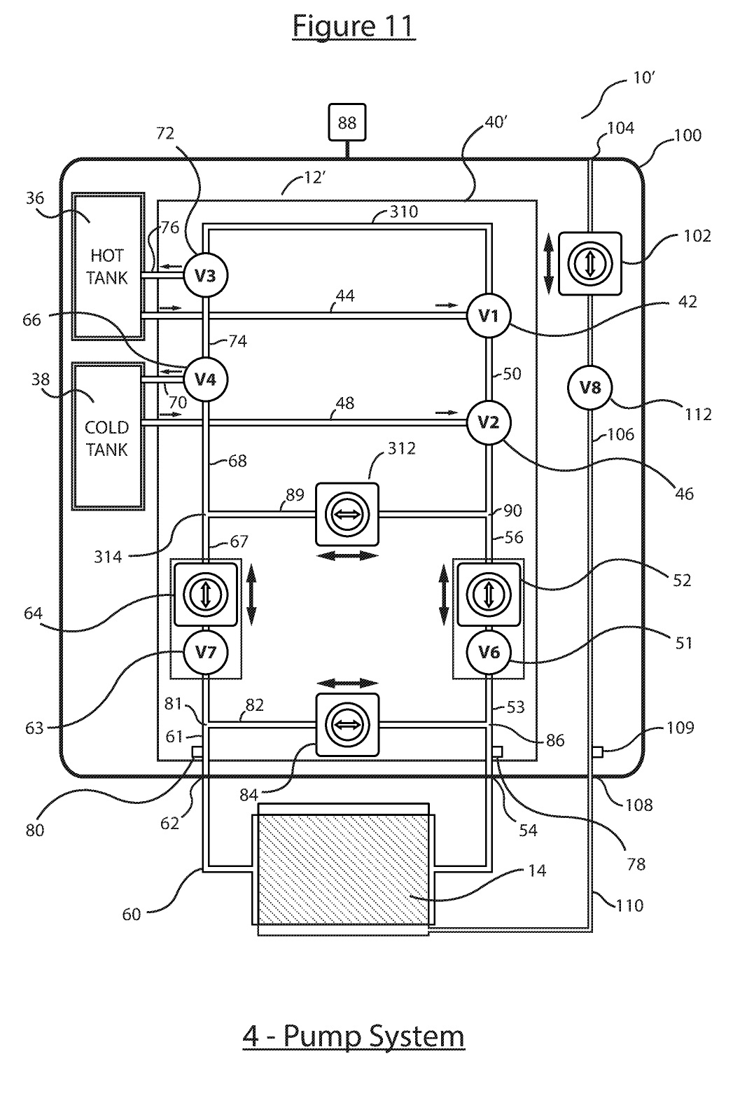

An assembly 10′ according to another embodiment of the invention is shown in FIG. 11. The features of the embodiment of FIG. 11 correspond to the features of the embodiment of FIG. 1 except for the differences outlined below. In particular, the pressure fluid supply system 100 is the same.

In the embodiment of FIG. 11, the conduit 87 is omitted. Accordingly, the flow circulation valve 51 and the return circulation valve 63 can in this embodiment be two-port valves instead of three-port valves.

In addition, the hot tank return valve 72 and the hot tank flow valve 42 can be three-port valves instead of two-port valves, and the second output port of the hot tank return valve 72 can be coupled to the second input port of the hot tank flow valve 42 by a conduit 310. The conduit 310 can in this embodiment be considered to be a first recycling conduit as it can direct thermoregulation fluid from the hot tank return valve to the hot tank flow valve without passing through the hot tank itself.

In this embodiment, the hot tank 36, the cold tank 38, the hot tank return valve 72, the hot tank flow valve 42, the cold tank return valve 66, the cold tank flow valve 46, and the conduits 76, 44, 70 and 48 can be considered to provide a regulated fluid supply unit in which the hot tank return valve, and the cold tank return valve 66 provide input junctions thereto, and the hot tank flow valve 42, and the cold tank flow valve 46 provide output junctions therefrom. As described above, by operation of the valves 72, 66, 42, 46, thermoregulation fluid can be selectively directed into the hot and cold tanks from the regulated fluid supply unit input junctions, and out of the hot and cold tanks by the regulated fluid supply unit output junctions or can be directed past the hot and cold tanks. The first recycling conduit 310 is able to direct fluid from the regulated fluid supply unit input junction 72 to the regulated fluid supply unit output junction 42 whereby to recycle thermoregulation fluid within the thermoregulation fluid supply system. This can be advantageous where fluid is being circulated in the thermoregulation fluid supply system 12′ without being passed to the hot or cold tanks.

In addition, in this embodiment, the tank circulation valve 65 is omitted and the conduit 89 includes a pack bypass pump 312 coupled to it. Accordingly, in this embodiment, the conduit 89 is coupled to a junction 314 between the conduit 67 and 68 instead of being coupled to the tank circulation valve 65.

The pack bypass pump 312 is configured to prevent fluid flowing through the pack bypass pump 312 along conduit 89 except when the pump 312 is being operated to pump fluid therealong. In other words, when the pack bypass pump 312 is not operating, it blocks fluid from travelling along the conduit 89. In the preferred embodiment, this function is provided by the pack bypass pump being a geared pump. In this embodiment, the pack bypass pump 312 is reversible so as to be able to pump fluid in either direction along the conduit 89.

In some embodiments, in a similar manner to the pack bypass pump 312, the return pump and/or the feed pump can be configured so as to prevent fluid from passing through them except when they are operating to pump fluid. For example, like the pack bypass pump 312, they can be geared pumps. This can mean that in some embodiments, the return circulation valve 63 and the flow circulation valve 51 can be omitted altogether.

In some embodiments, the mixing system bypass pump 84 can be configured to prevent fluid from passing along the conduit 82 except when the mixing system bypass pump 84 is pumping fluid therealong. For example, it can be a geared pump.

In the present embodiment, the mixing system can be considered to include the input and output pumps, the valves 42, 46, 66 and 72, the pump 312, and the conduits 50, 56, 67, 68, 74, 89 and 310. However, as described above, the precise extent of the mixing system is not important.

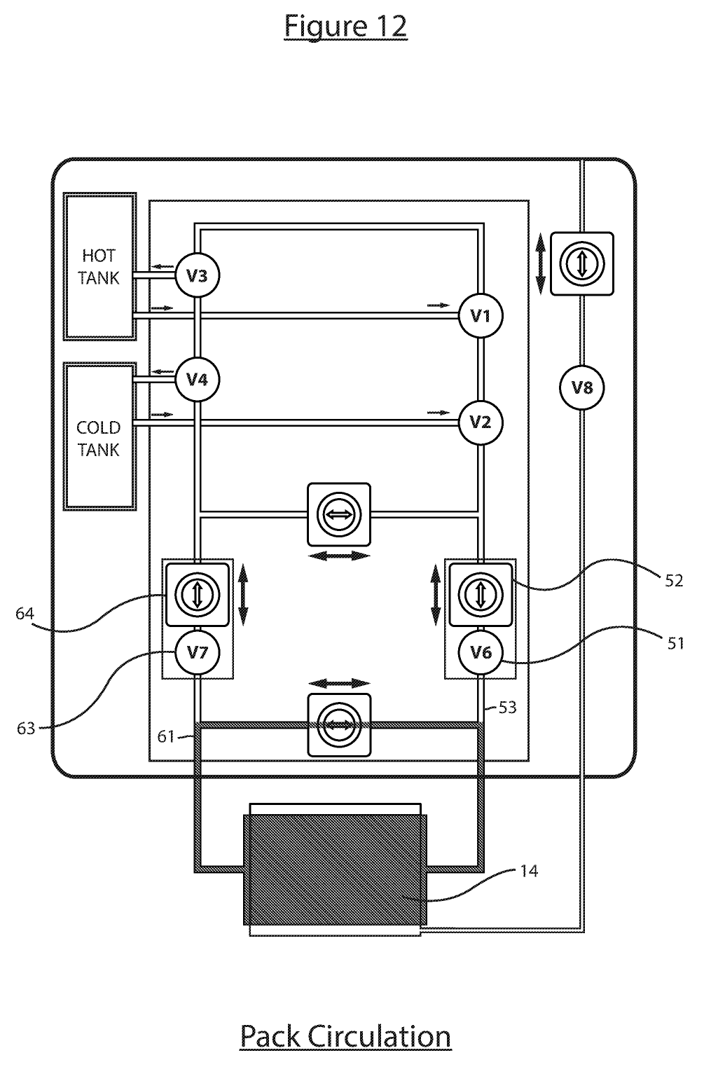

During operation of the embodiment of FIG. 11, as for the embodiment of FIG. 1, it is possible for thermoregulation fluid to be circulated around the pack 14 without passing into the regulated fluid supply. This is shown in FIG. 12 and operates in a similar manner to that shown and described with respect to FIG. 4. However, in embodiments in which the return circulation valve 63 and the flow circulation valve 51 are omitted, thermoregulation fluid can be obstructed in conduits 61 and 53 by the pumps 64 and 52 being in a non-operating configuration.

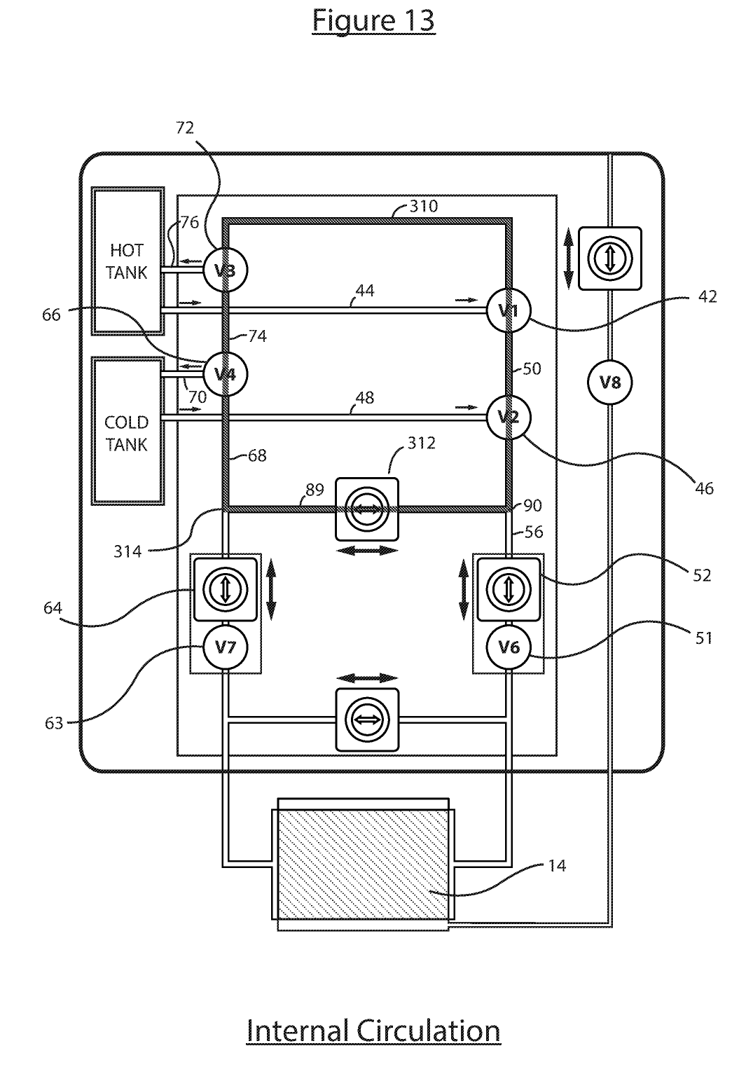

The assembly 10′ can also be operated in an internal circulation mode as shown in FIG. 13. As for the mode of operation shown in FIG. 5, in this situation, fluid is circulated within the mixing system without passing to the pack 14. In this embodiment, the dosing or feed pump 52 and return pump 64 are not operating. In the embodiments in which the flow circulation valve 51 and the return circulation valve 63 are present, these are in the closed position. Accordingly, fluid does generally not flow closer to the pack 14 than the second recycling conduit 89.

The dosing or flow pump 52 and the return pump 64 do not need to be operated in this embodiment since the internal circulation is provided by the pack bypass pump 312. While this can be operated in either direction, for internal circulation it is preferred to operate the pump 312 to pump fluid in the direction from junction 90 to junction 314 as this makes it easier to add and remove fluid from the hot and/or cold tanks.

Because of the presence of the first recycling conduit 310, thermoregulation fluid can pass from the pump 312 to junction 314, along conduit 68, 74, 310 and 50. Fluid can then pass to the junction 90 and return to the pump 312 along conduit 89. In order for this to be achieved, the cold tank return valve 66 is configured to allow fluid from conduit 68 to pass to conduit 74 but not conduit 70. The hot tank return valve 72 is configured to allow fluid from conduit 74 to pass to conduit 310 but not to conduit 76. The hot tank flow valve 42 is configured to allow fluid from conduit 310 to pass to conduit 50 but not to accept fluid from conduit 44. The cold tank flow valve 46 is configured to allow fluid from conduit 50 to pass to conduit 56 but not to accept fluid from conduit 48.

As for the internal circulation of FIG. 5, the internal circulation of FIG. 13 can be used advantageously to bring thermoregulation fluid to a desired temperature. This can be performed by appropriate operation of the hot and cold tank flow and return valves to allow thermoregulation fluid to be passed to the hot and/or cold tanks and/or to allow thermoregulation fluid from the hot and/or cold tanks to be added to the circulating thermoregulation fluid. This can be repeated at intervals in order gradually to introduce hot and/or cold thermoregulation fluid to the circulating thermoregulation fluid in order slowly to bring the thermoregulation fluid to a desired temperature.

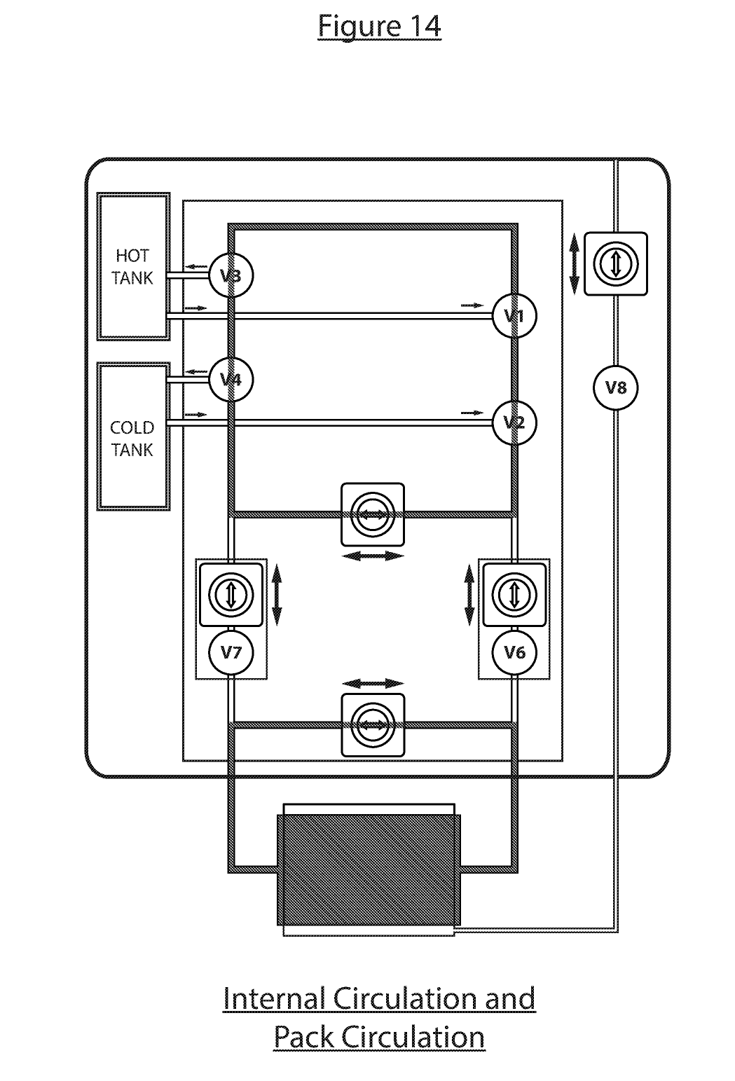

The internal circulation and the pack circulation can be operated simultaneously in order to circulate the existing pack fluid while further fluid is brought to the correct temperature. This is shown in FIG. 14 and shows the simultaneous operation of the operations of FIGS. 12 and 13.

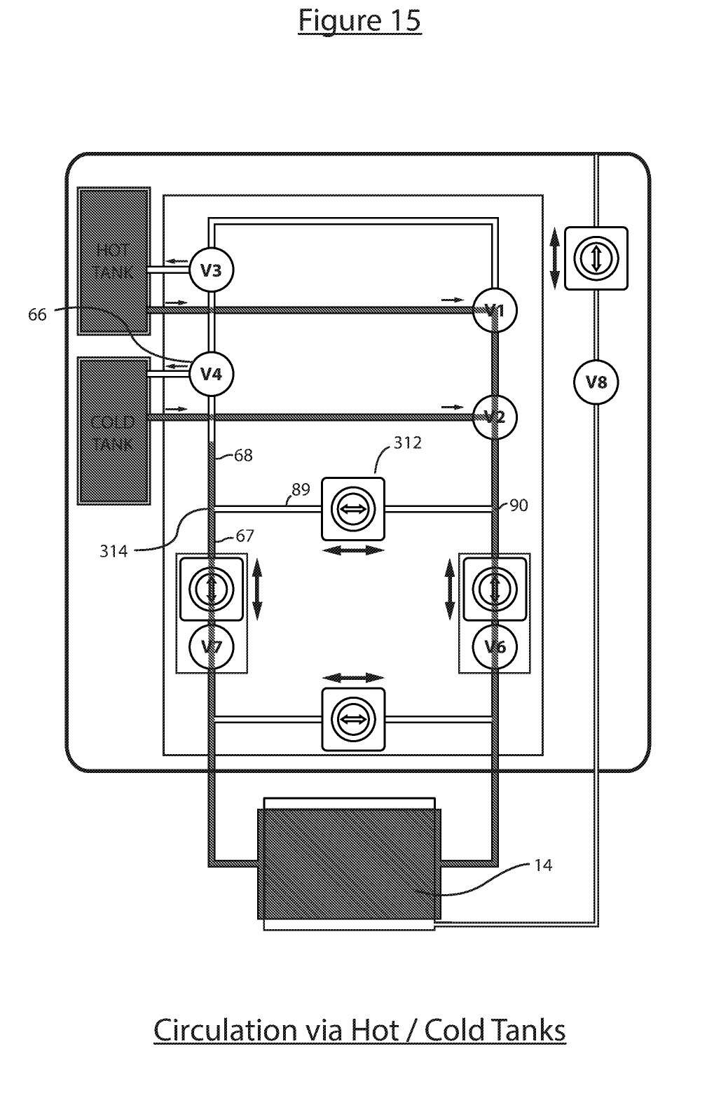

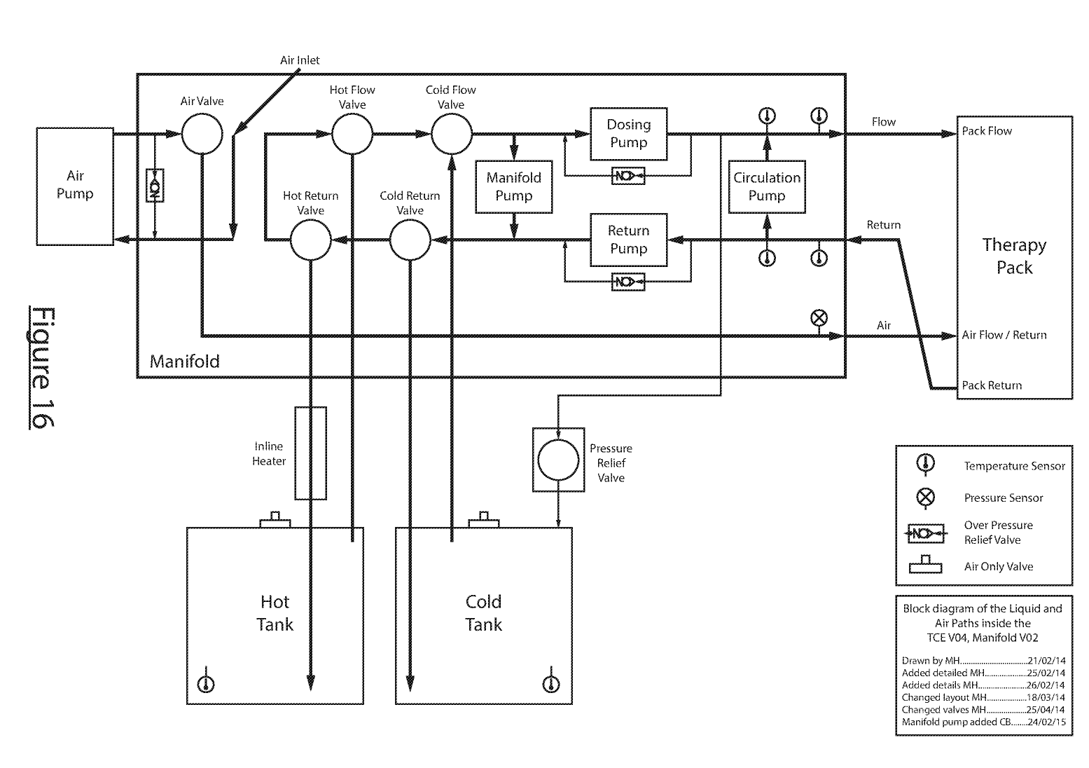

FIG. 15 shows the assembly 10′ being operated to provide thermoregulation fluid directly to the pack from the mixing system, in particular from the hot and/or cold tanks. This method of operation is similar to that described in respect of FIG. 7. However, in this embodiment, there is no tank circulation valve 65. Thermoregulation fluid is prevented from passing to junction 90 from junction 314 by the pack bypass pump 312 being in a non-operating position and thereby blocking the passage of fluid along conduit 89. The thermoregulation fluid is free to flow from conduit 67 into conduit 68.

Furthermore, in order to recycle fluid without passing it to the hot and/or cold tanks, it is possible to transition the cold tank return valve 66 into a fully closed configuration and to operate the pack bypass pump 312 to pump fluid in the direction from junction 314 to junction 90 thereby serving as a recycling pump. In this way, fluid from the pack 14 is passed from junction 314 to junction 90 to be recycled without passing to the regulated fluid supply unit. However, at intervals, the operation can be transitioned to that described above in respect of FIG. 15 in order to remove some thermoregulation fluid from the circulating fluid and/or to add some thermoregulation fluid from the regulated fluid supply in order to change the temperature of the thermoregulation fluid being supplied to the pack.

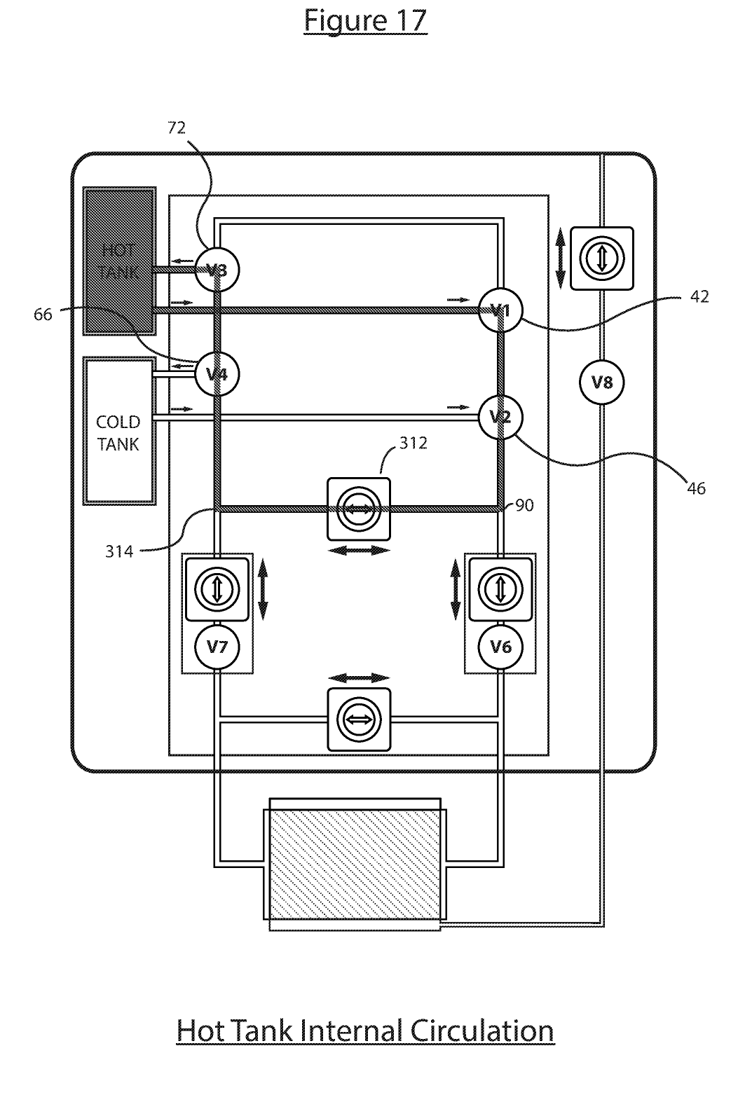

As shown in FIG. 17, it is also possible by appropriate operation of the valves 42, 46, 66 and 72, and by operating the pack bypass pump 312 to pump fluid from junction 90 to junction 314 to operate a hot tank internal circulation.

This allows the thermoregulation fluid in the hot tank to be mixed as the thermoregulation fluid supply system prepares before treatment. This prevents an inconsistent temperature in the hot tank, prevents a temperature gradient occurring and enables the hot tank to get to temperature more efficiently and deliver a more accurate temperature of thermoregulation fluid.

A corresponding cold tank internal circulation is also possible.

In embodiments, the hot tank and cold tank can be interchanged.

All optional and preferred features and modifications of the described embodiments and dependent claims are usable in all aspects of the invention taught herein. Furthermore, the individual features of the dependent claims, as well as all optional and preferred features and modifications of the described embodiments are combinable and interchangeable with one another.
The disclosures in United Kingdom patent application numbers GB 1413601.4 and 1505218.6, from which this application claims priority, and in the abstract accompanying this application are incorporated herein by reference.

1-15. (canceled)

16. A thermoregulation system including:
a. an output port configured to be coupled to an input of a personal thermoregulation pack,
b. an input port configured to be coupled to an output of the personal thermoregulation pack,
c. a thermoregulation fluid supply system configured to supply thermoregulation fluid to the output port,
d. an output pump configured to pump the thermoregulation fluid from the output port,
e. an input pump configured to pump the thermoregulation fluid into the input port, and
f. a pressure control system configured to pressurize a pressurizing element of the personal thermoregulation pack, wherein the pressurization of the pressurizing element is independent of the pumping of the input and output pumps.

17. The thermoregulation system of claim 16 wherein each of the input and output pumps is configured to pump the thermoregulation fluid at a rate independent of the other of the input and output pumps.

18. The thermoregulation system of claim 16 wherein the system is configured to independently control:
a. a pressure with which a coupled personal thermoregulation pack is pressed against a user,
b. a flow rate of the thermoregulation fluid to the output port,
c. a flow rate of the thermoregulation fluid into the input port, and
d. a temperature of the thermoregulation fluid exiting the output port.

19. The thermoregulation system of claim 16 wherein the pressure control system includes:
a. a pressure fluid outlet port configured to be coupled to the pressurizing element of the personal interface pack, and
b. a pressure fluid pump configured to pump pressure fluid into and/or out of the pressure fluid outlet port.

20. The thermoregulation system of claim 16 further including a temperature control unit configured to provide the thermoregulation fluid at a predetermined thermoregulation temperature.

21. The thermoregulation system of claim 20 wherein the temperature control unit is configured to control the temperature of the provided thermoregulation fluid independently of:
a. a pressure with which a coupled personal thermoregulation pack is pressed against a user,
b. a flow rate of the thermoregulation fluid to the output port, and
c. a flow rate of the thermoregulation fluid into the input port.

22. The thermoregulation system of claim 20 further including a temperature control unit bypass conduit configured to be selectively activated to cause the thermoregulation fluid pumped into the input port to be directed to the output port without passing through the temperature control unit.

23. The thermoregulation system of claim 22 further including a temperature control unit bypass pump:
a. coupled to the temperature control unit bypass conduit, and
b. configured to pump the thermoregulation fluid along the temperature control unit bypass conduit and through the thermoregulation pack.

24. The thermoregulation system of claim 20 wherein:
a. the temperature control unit includes:
(1) a hot fluid supply configured to supply hot thermoregulation fluid,
(2) a cold fluid supply configured to supply cold thermoregulation fluid,
b. the output pump is configured to selectively pump:
(1) hot thermoregulation fluid from the hot fluid supply,
(2) cold thermoregulation fluid from the cold fluid supply, and
(3) a mixture of hot thermoregulation fluid from the hot fluid supply and cold thermoregulation fluid from the cold fluid supply,
from the output port,
c. the input pump is configured to selectively pump the thermoregulation fluid into the input port to:
(1) the hot fluid supply,
(2) the cold fluid supply, and
(3) both the hot fluid supply and the cold fluid supply.

25. The thermoregulation system of claim 16 further including a personal interface pack configured to couple to the input port and to the output port.

26. The thermoregulation system of claim 16 further including a system control unit configured to:
a. operate the output pump to pump the thermoregulation fluid from the output port, and
b. operate the input pump to pump the thermoregulation fluid into the input port.

27. The thermoregulation system of claim 16 further including:
a. a hot fluid supply configured to supply hot thermoregulation fluid,
b. a cold fluid supply configured to supply cold thermoregulation fluid,
c. an output fluid path conveying hot thermoregulation fluid from the hot fluid supply and cold thermoregulation fluid from the cold fluid supply through the output pump and the output port,
d. an input fluid path conveying thermoregulation fluid from the input port through the input pump,
e. a first bypass fluid path extending:
(1) from a location along the input fluid path between the input port and the input pump,
(2) to a location along the output fluid path between the output pump and the output port.

28. The thermoregulation system of claim 27 further including a bypass pump situated along the first bypass fluid path, whereby the bypass pump may convey thermoregulation fluid pumped into the input port to the output port without passing through the input pump.

29. The thermoregulation system of claim 27 further including a second bypass fluid path extending:
a. from a location along the input fluid path downstream from the first bypass fluid path and upstream from the input pump,
b. to a location along the output fluid path downstream from the output pump and upstream from the first bypass fluid path.

30. The thermoregulation system of claim 29 further including a third bypass fluid path extending:
a. from a location along the input fluid path downstream from the input pump,
b. to a location along the output fluid path upstream from the output pump.

31. The thermoregulation system of claim 30 wherein each of the second and third bypass fluid paths include at least one valve therealong, wherein the valves are configured to selectively allow or prohibit flow of thermoregulation fluid between the input fluid path and the output fluid path.

32. The thermoregulation system of claim 29 further including:
a. a first valve connecting the second bypass fluid path to the input fluid path, and
b. a second valve connecting the second bypass fluid path to the output fluid path.

33. A method of operating the thermoregulation system of claim 16, the method including the steps of:
a. pumping the thermoregulation fluid from the output port,
b. pumping the thermoregulation fluid into the input port, and
c. pressurizing the pressurizing element of the personal thermoregulation pack.

34. The method of claim 33 wherein the thermoregulation fluid is pumped from the output port to provide a desired volume of thermoregulation fluid to the personal thermoregulation pack before pressurizing the pressurizing element of the personal thermoregulation pack.
